(19) United States
(12) Patent Application Publication
(10) Pub. No.: US 2003/0045722 A1
(43) Pub. Date: Mar. 6, 2003

(54) Processes for preparing anhydrous and hydrate forms of antihistaminic piperidine derivatives, polymorphs and pseudomorphs thereof

(76) Inventors: Daniel R. Henton, Midland, MI (US); Frederick J. McCarty, Hilton Head Island, SC (US); Susan I. Tripp, Lee's Summit, MO (US); Jill E. Dewitt, Kansas City, MO (US); Scott E. Peterson, Raymore, MO (US); William H. Streng, Peculiar, MO (US)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 10/125,094

(22) Filed: Apr. 18, 2002

Related U.S. Application Data
(63) Continuation of application No. 09/653,082, filed on Aug. 31, 2000.
Application No. 09/653,082 is a continuation of application No. 09/276,069, filed on Mar. 25, 1999, now abandoned.
Application No. 09/276,069 is a continuation of application No. 08/899,843, filed on Jul. 24, 1997, now abandoned.
Application No. 08/899,843 is a continuation-in-part of application No. 08/818,087, filed on Mar. 14, 1997, now abandoned.
Application No. 08/818,087 is a continuation of application No. 08/442,460, filed on May 16, 1995, now abandoned.
Application No. 08/442,460 is a division of application No. 08/417,161, filed on Apr. 11, 1995, now abandoned.
Application No. 08/417,161 is a continuation-in-part of application No. 08/245,731, filed on May 18, 1994, now abandoned.

(51) Int. Cl. A61K 31/445; C07D 211/34

(52) U.S. Cl. 546/238; 514/317

(57) ABSTRACT

The present invention is related to novel processes for preparing anhydrous and hydrated forms of piperidine derivatives, polymorphs and pseudomorphs thereof of the formulas

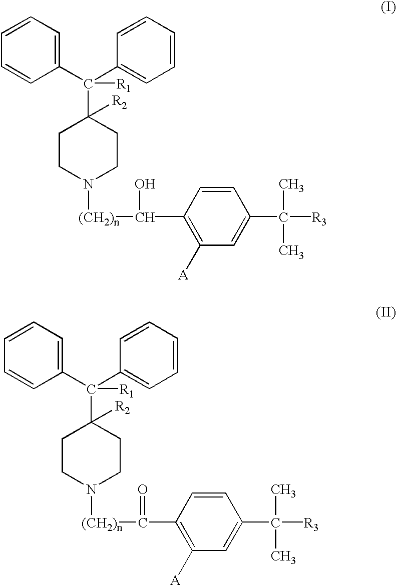

which are useful as antihistamines, antiallergic agents and bronchodilators.

This is a Continuation-In-Part Application of patent application Ser. No. 08/818,087, filed Mar. 14,1997 which is a Continuation Application of patent application Ser. No. 08/442,460, filed May 16, 1995, now abandoned, which is a Divisional Application of patent application Ser. No. 08/417,161, filed Apr. 11, 1995, now abandoned, which is a Continuation-In-Part Application of patent application Ser. No. 08/245,731, filed May 18, 1994, now abandoned.

The present invention is related to novel processes for preparing anhydrous and hydrated forms of piperidine derivatives, polymorphs and pseudomorphs thereof which are useful as antihistamines, antiallergic agents and bronchodilators [U.S. Pat. No. 4,254,129, Mar. 3, 1981, U.S. Pat. No. 4,254,130, Mar. 3, 1981 and U.S. Pat. No. 4,285,958, Apr. 25, 1981].

### SUMMARY OF THE INVENTION

The present invention provides a process for preparing anhydrous, pharmaceutically acceptable acid addition salts of piperidine derivatives of the formulas wherein

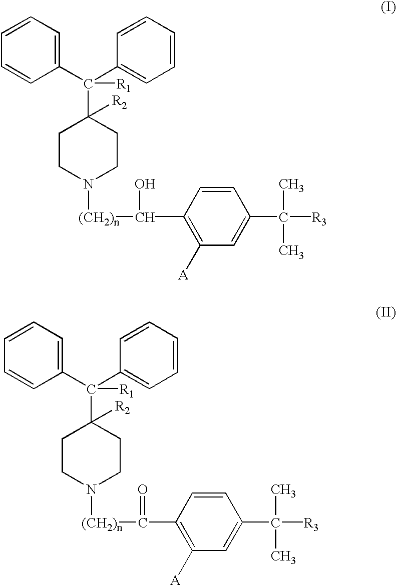

R1 represents hydrogen or hydroxy;

R₂ represents hydrogen; or

R₁ and R₂ taken together form a second bond between the carbon atoms bearing R₁ and R₂;

n is an integer of from 1 to 5;

R₃ is -CH₂OH, -COOH or -COOalkyl wherein the alkyl moiety has from 1 to 6 carbon atoms and is straight or branched;

each of A is hydrogen or hydroxy; and

pharmaceutically acceptable salts and individual optical isomers thereof, comprising subjecting the corresponding hydrated, pharmaceutically acceptable acid addition salt to an azeotropic distillation.

In addition, the present invention also provides a process for preparing anhydrous, pharmaceutically acceptable acid addition salts of piperidine derivatives of the formula

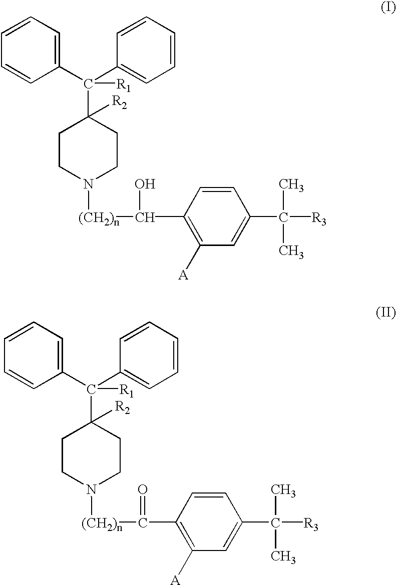

wherein

R₁ represents hydrogen or hydroxy;

R₂ represents hydrogen; or

R₁ and R₂ taken together form a second bond between the carbon atoms bearing R₁ and R₂;

n is an integer of from 1 to 5;

R₃ is -CH₂OH, -COOH or -COOalkyl wherein the alkyl moiety has from 1 to 6 carbon atoms and is straight or branched;

each of A is hydrogen or hydroxy; and

pharmaceutically acceptable salts and individual optical isomers thereof, comprising subjecting the corresponding hydrated, pharmaceutically acceptable acid addition salt to a water-minimizing recrystallization.

In addition, the present invention provides a process for preparing the hydrated, pharmaceutically acceptable acid addition salts of piperidine derivatives of the formula

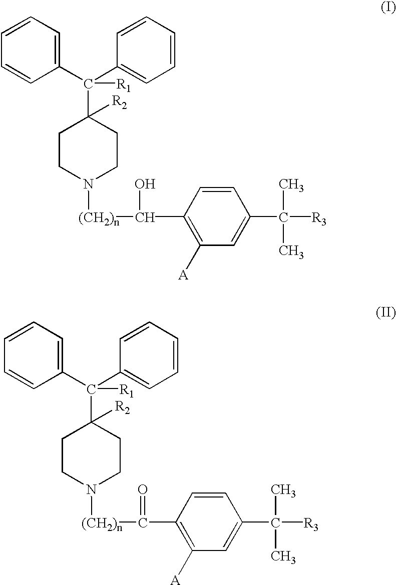

wherein

R₁ represents hydrogen or hydroxy;

R₂ represents hydrogen; or

R₁ and R₂ taken together form a second bond between the carbon atoms bearing R₁ and R₂;

n is an integer of from 1 to 5;

R₃ is -CH₂OH, -COOH or -COOalkyl wherein the alkyl moiety has from 1 to 6 carbon atoms and is straight or branched;

each of A is hydrogen or hydroxy; and

pharmaceutically acceptable salts and individual optical isomers thereof, comprising subjecting the corresponding anhydrous, pharmaceutically acceptable acid addition salts to an aqueous recrystallization.

In addition, the present invention provides pseudomorphs of hydrated 4-[4-[4-α,α-(hydroxydiphenylmethyl)-1-piperidinyl]-1-hydroxybutyl]-dimethylbenzeneacetic acid free base, designated herein as Forms I′, II′, III′, IV′, V′, VIII′ and IX′ and a pseudomorph of anhydrous 4-[4-[4-α,α-(hydroxydiphenylmethyl)-1-piperidinyl]-1-hydroxybutyl]-dimethylbenzeneacetic acid free base, designated herein as Form VII′.

The Form I′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: a first endothermic transition having a peak termperature of 100.7° C. which is associated with the loss of water, followed by two exothermic transitions: the first with an onset of approximately 144.3° C. and the second with an onset of approximately 180.8° C., followed by a final melt onset at approximately 226.9° C. and an X-ray powder diffraction pattern essentially as shown in Table 1 wherein the XRPD patterns were measured using a powder diffractometer equipped with a copper X-ray tube source. The sample was illuminated with copper Kal radiation and XRPD data were collected from 2° to 42°2θ. (intensities may vary radically due to preferred orientation).

TABLE 1

| D-Space, Angstroms | Intensity, I/Iₒ, % |
|---|---|
| 24.153 | 51 |
| 11.893 | 11 |
| 9.066 | 36 |
| 7.562 | 33 |
| 7.281 | 19 |
| 6.371 | 17 |
| 6.154 | 14 |
| 5.444 | 15 |
| 4.913 | 100 |
| 4.793 | 99 |
| 4.498 | 61 |
| 4.286 | 16 |
| 4.209 | 18 |
| 4.047 | 32 |
| 3.895 | 15 |
| 3.704 | 24 |
| 3.435 | 20 |
| 3.331 | 15 |
| 3.290 | 14 |
| 3.278 | 13 |
| 3.179 | 15 |
| 3.111 | 11 |
| 2.841 | 14 |
| 2.751 | 12 |

The Form II′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: first and second endothermic transitions at less than 100° C., both associated with the loss of water, followed by a final melt onset at approximatley 151.3° C. and an X-ray powder diffraction pattern essentially as shown in Table 2 wherein the XRPD patterns were measured using a powder diffractometer equipped with a copper X-ray tube source. The sample was illuminated with copper Kα1 radiation and XRPD data were collected from 2° to 42°2θ. (intensities may vary radically due to preferred orientation).

TABLE 2

| D-Space, Angstroms | Intensity, I/Iₒ, % |
|---|---|
| 12.961 | 12 |
| 10.530 | 66 |
| 9.351 | 26 |
| 8.165 | 68 |
| 6.677 | 38 |
| 6.475 | 26 |
| 5.560 | 41 |
| 5.387 | 60 |
| 5.215 | 100 |
| 4.983 | 26 |
| 4.666 | 23 |
| 4.469 | 19 |
| 4.418 | 72 |
| 4.314 | 26 |
| 4.229 | 19 |
| 4.158 | 67 |
| 3.985 | 30 |
| 3.921 | 30 |
| 3.819 | 47 |
| 3.358 | 18 |
| 2.940 | 21 |

The Form III′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: a first endothermic transition having a onset temperature of 130.1° C. which is associated with the loss of water, followed by an exothermic transition with an onset of approximately 166.2° C., followed by a final melt onset at approximately 225.9° C. and an X-ray powder diffraction pattern essentially as shown in Table 3 wherein the XRPD patterns were measured using a powder diffractometer equipped with a copper X-ray tube source. The sample was illuminated with copper Kα₁ radiation and XRPD data were collected from 2° to 42°2θ. (intensities may vary radically due to preferred orientation).

TABLE 3

| D-Space, Angstroms | Intensity, I/Iₒ, % |
|---|---|
| 20.108 | 100 |
| 10.142 | 31 |
| 8.450 | 19 |
| 7.856 | 20 |
| 7.616 | 16 |
| 7.405 | 17 |
| 7.057 | 38 |
| 6.771 | 26 |
| 6.515 | 31 |
| 5.435 | 77 |
| 5.320 | 44 |
| 5.073 | 65 |
| 4.784 | 60 |
| 4.526 | 32 |
| 4.328 | 24 |
| 4.274 | 48 |
| 4.080 | 55 |
| 3.925 | 42 |
| 3.277 | 41 |

The Form IV′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: a first endothermic transition at less than 100° C. which is associated with the loss of water, followed by a second endothermic transition having an onset temperature of 154.3° C., followed by an exothermic transition with an onset of approximately 186.6° C., followed by a final melt onset at approximately 229.1° C. and an X-ray powder diffraction pattern essentially as shown in Table 4 wherein the XRPD patterns were measured using a powder diffractometer equipped with a copper X-ray tube source. The sample was illuminated with copper Kα₁ radiation and XRPD data were collected from 2° to 42°2θ. (intensities may vary radically due to preferred orientation).

TABLE 4

| D-Space, Angstroms | Intensity, I/Iₒ, % |
|---|---|
| 20.367 | 100 |
| 10.529 | 61 |
| 9.629 | 13 |
| 8.304 | 9 |
| 7.689 | 11 |
| 7.020 | 9 |
| 6.030 | 12 |
| 5.462 | 11 |
| 5.257 | 17 |
| 5.056 | 20 |
| 4.960 | 7 |
| 4.869 | 7 |
| 4.645 | 9 |
| 4.572 | 27 |
| 4.392 | 6 |
| 4.239 | 7 |
| 4.136 | 19 |
| 4.019 | 12 |
| 3.394 | 6 |

The Form V′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: a first endothermic transition less than 100° C. which is associated with the loss of water, followed by a second endothermic transition having an onset temperature of 143.9° C., followed by an exothermic transition with a peak of approximately 174.7° C., followed by a final melt onset at approximately 227.4° C. and an X-ray powder diffraction pattern essentially as shown in Table 5 wherein the XRPD patterns were measured using a powder diffractometer equipped with a copper X-ray tube source. The sample was illuminated with copper Kα₁ radiation and XRPD data were collected from 2° to 42°2θ. (intensities may vary radically due to preferred orientation).

TABLE 5

| D-Space, Angstroms | Intensity, I/Iₒ, % |
|---|---|
| 21.946 | 60 |
| 10.995 | 53 |
| 10.111 | 36 |
| 9.586 | 18 |
| 9.076 | 21 |
| 8.506 | 18 |
| 8.119 | 20 |
| 7.695 | 29 |
| 7.504 | 25 |
| 6.413 | 24 |
| 6.218 | 59 |
| 5.588 | 42 |
| 5.035 | 38 |
| 4.786 | 23 |
| 4.636 | 41 |
| 4.534 | 100 |
| 4.370 | 19 |
| 4.226 | 19 |
| 3.989 | 37 |

The Form VIII′ free base pseudomorph of hydrated 4-[4-[4-α,α-(hydroxydiphenylmethyl)-1-piperidinyl]-1-hydroxybutyl]-dimethylbenzeneacetic acid free base may be identified by the following characteristics: a first endothermic transition having a peak temperature of 146.9° C. followed by a second endothermic transition having a peak temperature of 170.95° C., followed by a final melt onset at approximately 227.1° C. and an X-ray powder diffraction pattern essentially as shown in Table 6 wherein the XRPD patterns were measured using a powder diffractometer equipped with a copper X-ray tube source. The sample was illuminated with copper Kα₁ radiation and XRPD data were collected from 2° to 42° 2θ. (intensities may vary radically due to preferred orientation).

TABLE 6

| D-Space, Angstroms | Intensity, I/Iₒ, % |
|---|---|
| 22.459 | 100 |
| 11.300 | 17 |
| 9.227 | 61 |
| 7.530 | 23 |
| 6.377 | 11 |
| 5.614 | 28 |
| 525.5 | 41 |
| 5.379 | 22 |
| 5.154 | 17 |
| 4.912 | 11 |
| 4.685 | 15 |
| 4.534 | 17 |
| 4.294 | 8 |
| 4.131 | 9 |
| 3.875 | 8 |
| 3.767 | 10 |

The Form IX′ free base pseudomorph of hydrated 4-[4-[4-α,α-(hydroxydiphenylmethyl)- 1-piperidinyl]-1-hydroxybutyl]-dimethylbenzeneacetic acid free base may be identified by the following characteristics: a first endothermic transition having a peak termperature of 96.95° C., followed by a second endothermic transition having a peak temperature of 135.12° C., followed by a final melt onset at approximately 229.84° C. and an X-ray powder diffraction pattern essentially as shown in Table 7 wherein the XRPD patterns were measured using a powder diffractometer equipped with a copper X-ray tube source. The sample was illuminated with copper Kα₁ radiation and XRPD data were collected from 2° to 42° 2θ. (intensities may vary radically due to preferred orientation).

TABLE 7

| D-Space, Angstroms | Intensity, I/Iₒ, % |
|---|---|
| 20.100 | 100 |
| 9.073 | 23 |
| 8.676 | 49 |
| 7.998 | 23 |
| 7.911 | 28 |
| 7.532 | 16 |
| 6.910 | 10 |
| 6.769 | 22 |
| 6.021 | 15 |
| 5.486 | 94 |
| 5.066 | 30 |
| 5.000 | 20 |
| 4.917 | 33 |
| 4.887 | 34 |
| 4.816 | 35 |
| 4.593 | 41 |
| 4.522 | 58 |
| 4.312 | 10 |
| 4.233 | 22 |
| 4.101 | 17 |
| 4.058 | 28 |
| 3.941 | 14 |

The Form VII′ free base pseudomorph of anhydrous ⁴-[⁴-[⁴-α,α-(hydroxydiphenylmethyl)-1-piperidinyl]-1-hydroxybutyl]-dimethylbenzeneacetic acid free base may be identified by the following characteristics: one major endothermic melt onset at approximately 221.1° C. and an X-ray powder diffraction pattern essentially as shown in Table 8 wherein the XRPD patterns were measured using a powder diffractometer equipped with a copper X-ray tube source. The sample was illuminated with copper Kα₁ radiation and XRPD data were collected from 2° to 42° 2θ. (intensities may vary radically due to preferred orientation).

TABLE 8

| D-Space, Angstroms | Intensity, I/Iₒ, % |
|---|---|
| 23.561 | 100 |
| 11.545 | 53 |
| 8.404 | 36 |
| 7.267 | 9 |
| 7.112 | 14 |
| 6.579 | 15 |
| 6.094 | 16 |
| 5.756 | 18 |
| 5.408 | 28 |
| 5.336 | 24 |
| 5.060 | 42 |
| 4.679 | 13 |
| 4.615 | 32 |
| 4.497 | 18 |
| 4.246 | 13 |
| 4.155 | 21 |
| 4.074 | 14 |
| 4.045 | 15 |
| 3.539 | 18 |
| 3.287 | 22 |
| 3.114 | 12 |

In addition, the present invention provides processes for preparing polymorphs of anhydrous 4-[4-[4-α,α-(Hydroxydiphenylmethyl)-1-piperidinyl]-1-hydroxybutyl]-dimethylbenzeneacetic acid hydrochloride designated herein as Form I and Form III and processes for preparing psuedomorphs of hydrated 4-[4-[4-(Hydroxydiphenylmethyl)-1-piperidinyl]-1-hydroxybutyl]-α,α-dimethylbenzeneacetic acid hydrochloride designated herein as Form II and Form IV.

The Form I anhydrous hydrochloride salt polymorph may be identified by the following characteristics: a visual melting point (capillary tube) in the range of about 196-201° C.; a melt endotherm with extrapolated onset in the range of about 195-199° C. as determined by differential scanning calorimetry; and an X-ray powder diffraction pattern essentially as shown in Table 9 wherein the XRPD patterns were measured using a powder diffractometer equipped with a Co X-ray tube source. The sample was illuminated with Co Kα₁ radiation and XRPD data were collected from 5 to 55° 2θ. (intensities may vary radically due to preferred orientation).

TABLE 9

| D-Space, Angstroms | Intensity, I/Iₒ, % |
|---|---|
| 11.8 | 30 |
| 7.3 | 30 |
| 6.3 | 65 |
| 5.9 | 35 |
| 5.0 | 45 |
| 4.8 | 100 |
| 4.4 | 45 |
| 3.9 | 60 |
| 3.8 | 75 |
| 3.7 | 30 |

The Form III anhydrous hydrochloride salt polymorph may be identified by the following characteristics: a visual melting point (capillary tube) in the range of about 166-171° C.; a broad endotherm below about 90° C., a melt endotherm with an extrapolated onset of about 166° C. as determined by differential scanning calorimetry; and an X-ray powder diffraction pattern essentially as shown in Table wherein the XRPD patterns were measured using a powder diffractometer equipped with a Co X-ray tube source. The sample was illuminated with Co Kaα₁ radiation and XRPD data were collected from 5 to 55° 2θ. (intensities may vary radically due to preferred orientation).

TABLE 10

| D-Space, Angstroms | Intensity, I/Iₒ, % |
|---|---|
| 9.0 | 95 |
| 4.9 | 100 |
| 4.8 | 35 |
| 4.6 | 25 |
| 4.5 | 25 |
| 3.7 | 25 |

The Form II hydrated hydrochloride salt pseudomorph may be identified by the following characteristics: a visual melting point (capillary tube) in the range of about 100-105° C.; a large broad endotherm below about 100° C. and a small endothermic peak (about 2 joules/gram) with extrapolated onsets in the range of about 124-126° C. as determined by differential scanning calorimetry; and an X-ray powder diffraction pattern essentially as shown in Table 11 wherein the XRPD patterns were measured using a powder diffractometer equipped with a Co X-ray tube source. The sample was illuminated with Co Kα₁ radiation and XRPD data were collected from 5 to 55° 2θ. (intensities may vary radically due to preferred orientation).

TABLE 11

| D-Space, Angstroms | Intensity, I/Iₒ, % |
|---|---|
| 7.8 | 45 |
| 6.4 | 44 |
| 5.2 | 85 |
| 4.9 | 60 |
| 4.7 | 80 |
| 4.4 | 55 |
| 4.2 | 50 |
| 4.1 | 60 |
| 3.7 | 75 |
| 3.6 | 60 |
| 3.5 | 50 |

The Form IV hydrated hydrochloride salt pseudomorph may be identified by the following characteristics: a visual melting point (capillary tube) in the range of about 113-118° C.; two broad overlapping endotherms below about 100° C. and an additional endotherm with an extrapolated onset at approximately 146° C. as determined by differential scanning calorimetry and an X-ray powder diffraction pattern essentially as shown in Table 12 wherein the XRPD patterns were measured using a powder diffractometer equipped with a Co X-ray tube source. The sample was illuminated with Co Kα₁ radiation and XRPD data were collected from 5 to 55° 2θ. (intensities may vary radically due to preferred orientation).

TABLE 12

| D-Space, Angstroms | Intensity, I/Iₒ, % |
|---|---|
| 10.4 | 60 |
| 7.0 | 45 |
| 6.4 | 50 |
| 5.3 | 100 |
| 5.2 | 55 |
| 4.3 | 75 |
| 4.1 | 50 |
| 4.0 | 45 |
| 3.8 | 60 |
| 3.5 | 55 |

In addition, the present invention provides preferred non-buffered aqueous solution conformations of the various anhydrous and hydrated 4-[4-[4-α,α-(hydroxydiphenylmethyl)-1-piperidinyl]-1-hydroxybutyl]-dimethylbenzeneacetic acid free base and hydrochloride salts wherein the preferred aqueous solution conformations obtained are dependent on the initial solid conformation of the hydrated or anhydrous 4-[4-[4-(α,α-(hydroxydiphenylmethyl)-1-piperidinyl]-1-hydroxybutyl]-dimethylbenzeneacetic acid free base or hydrochloride salt which is dissolved.

As used herein, the preferred non-buffered aqeuous solution conformation derived from the dissolution of Form I′ hydrated 4-[4-[4-α,α-(hydroxydiphenylmethyl)-1-pipenidinyl]-1-hydroxybutyl]-dimethylbenzeneacetic acid free base is designated Form I′″;

As used herein, the preferred non-buffered aqeuous solution conformation derived from the dissolution of Form II′ hydrated 4-[4-[4-α,α-(hydroxydiphenylmethyl)-1-piperidinyl]-1-hydroxybutyl]-dimethylbenzeneacetic acid free base is designated Form II′″.

As used herein, the preferred non-buffered aqeuous solution conformation derived from the dissolution of Form II′ hydrated 4-[4-[4-α,α-(hydroxydiphenylmethyl)-1-piperidinyl]-1-hydroxybutyl]-dimethylbenzeneacetic acid free base is designated Form III′″.

As used herein, the preferred non-buffered aqeuous solution conformation derived from the dissolution of Form IV′ hydrated 4-[4-[4-α,α-(hydroxydiphenylmethyl)-1-piperidinyl]-1-hydroxybutyl]-dimethylbenzeneacetic acid free base is designated Form IV′″.

As used herein, the preferred non-buffered aqeuous solution conformation derived from the dissolution of Form V′ hydrated 4-[4-[4-α,α-(hydroxydiphenylmethyl)-1-piperidinyl]-1-hydroxybutyl]-dimethylbenzeneacetic acid free base is designated Form V′″.

As used herein, the preferred non-buffered aqeuous solution conformation derived from the dissolution of Form VIII′ hydrated 4-[4-[4-α,α-(hydroxydiphenylmethyl)-1-piperidinyl]-1-hydroxybutyl]-dimethylbenzeneacetic acid free base is designated Form VIII′″.

As used herein, the preferred non-buffered aqeuous solution conformation aid derived from the dissolution of Form IX′ hydrated 4-[4-[4-α,α-(hydroxydiphenylmethyl)-1-piperidinyl]-1-hydroxybutyl]-dimethylbenzeneacetic acid free base is designated Form IX′″.

As used herein, the preferred non-buffered aqeuous solution conformation derived from the dissolution of Form VII′ anhydrous 4-[4-[4 α,α-(hydroxydiphenylmethyl)-1-piperidinyl]-1-hydroxybutyl]-dimethylbenzeneacetic acid free base is designated Form VII′″.

As used herein, the preferred non-buffered aqeuous solution conformation derived from the dissolution of Form I anhydrous 4-[4-[4-α,α-(hydroxydiphenylmethyl)-1-piperidinyl]-1-hydroxybutyl]-dimethylbenzeneacetic acid hydrochloride salt is designated Form I″.

As used herein, the preferred non-buffered aqeuous solution conformation derived from the dissolution of Form III anhydrous 4-[4-[4-α,α-(hydroxydiphenylmethyl)-1-piperidinyl]-1-hydroxybutyl]-dimethylbenzeneacetic acid hydrochloride salt is designated Form III″.

As used herein, the preferred non-buffered aqeuous solution conformation derived from the dissolution of Form II hydrated 4-[4-[4-α,α-(hydroxydiphenylmethyl)-1-piperidinyl]-1-hydroxybutyl]-dimethylbenzeneacetic acid hydrochloride salt is designated Form II″.

As used herein, the preferred non-buffered aqeuous solution conformation derived from the dissolution of Form IV hydrated 4-[4-[4-α,α-(hydroxydiphenylmethyl)-1-piperidinyl]-1-hydroxybutyl]-dimethylbenzeneacetic acid hydrochloride salt is designated Form IV″.

Form I′″ preferred non-buffered aqeuous solution conformation of 4-[4-[4-α,α-(hydroxydiphenylmethyl)-1-piperidinyl]-1-hydroxybutyl]-dimethylbenzeneacetic acid may be identified by the following pH-Solubility Profile:

| pH | [M] |
|---|---|
| 1.27 | 5.98e⁻⁴ |
| 2.06 | 3.28e⁻³ |
| 1.58 | 1.32e⁻³ |
| 2.01 | 3.26e⁻³ |
| 2.69 | 4.95e⁻³ |
| 3.35 | 3.34e⁻³ |
| 3.49 | 2.87e⁻³ |
| 3.65 | 2.08e⁻³ |
| 4.06 | 1.07e⁻³ |
| 4.32 | 7.88e⁻⁴ |
| 4.51 | 6.56e⁻⁴ |
| 4.84 | 5.06e⁻⁴ |
| 5.98 | 3.67e⁻⁴ |
| 7.43 | 3.02e⁻⁴ |
| 7.83 | 3.51e⁻⁴ |
| 7.51 | 3.04e⁻⁴ |

Form I″ preferred non-buffered aqeuous solution conformation of 4-[4-[4-α,α-(hydroxydiphenylmethyl)-1-piperidinyl]-1-hydroxybutyl]-dimethylbenzeneacetic acid may be identified by the following pH-solubility Profile:

| pH | [M] |
|---|---|
| 1.09 | 4.99e⁻⁴ |
| 1.35 | 8.68e⁻⁴ |
| 2.05 | 2.89e⁻³ |
| 2.40 | 3.95e⁻³ |
| 2.60 | 4.32e⁻³ |
| 2.66 | 3.98e⁻³ |
| 2.68 | 3.97e⁻³ |
| 2.64 | 3.84e⁻³ |
| 2.68 | 3.81e⁻³ |
| 2.78 | 3.25e⁻³ |
| 2.77 | 3.12e⁻³ |
| 2.79 | 3.11e⁻³ |
| 2.75 | 3.17e⁻³ |
| 3.29 | 1.92e⁻³ |
| 4.28 | 1.04e⁻³ |
| 5.10 | 9.65e⁻⁴ |
| 6.80 | 1.05e⁻³ |

Form II″ preferred non-buffered aqeuous solution conformation of 4-[4-[4-α,α-(hydroxydiphenylmethyl)-1-piperidinyl]-1-hydroxybutyl]-dimethylbenzeneacetic acid may be identified by the following pH-solubility Profile:

| pH | [M] |
|---|---|
| 1.09 | 5.11e⁻⁴ |
| 1.38 | 8.56e⁻⁴ |
| 2.05 | 2.89e⁻³ |
| 2.35 | 3.97e⁻³ |
| 2.68 | 4.52e⁻³ |
| 2.74 | 4.14e⁻³ |
| 2.78 | 3.84e⁻³ |
| 2.98 | 3.18e⁻³ |
| 3.08 | 2.58e⁻³ |
| 3.43 | 1.61e⁻³ |
| 3.73 | 1.32e⁻³ |
| 4.35 | 1.11e⁻³ |
| 1.10 | 5.17e⁻⁴ |
| 1.40 | 1.00e⁻³ |
| 2.06 | 2.93e⁻³ |
| 2.38 | 3.98e⁻³ |
| 2.82 | 4.67e⁻³ |
| 2.86 | 4.54e⁻³ |
| 2.82 | 4.47e⁻³ |
| 3.03 | 3.41e⁻³ |
| 2.96 | 3.66e⁻³ |
| 4.69 | 8.49e⁻⁴ |

### DETAILED DESCRIPTION OF THE INVENTION

Pharmaceutically acceptable acid addition salts of the compounds of formula (I) and (II), both anhydrous and hydrated, are those of any suitable inorganic or organic acid. Suitable inorganic acids are, for example, hydrochloric, hydrobromic, sulfuric, and phosphoric acids. Suitable organic acids include carboxylic acids, such as, acetic, propionic, glycolic, lactic, pyruvic, malonic, succinic, fumaric, malic, tartaric, citric, cyclamic, ascorbic, maleic, hydroxymaleic, and dihydroxymaleic, benzoic, phenylacetic, 4-aminobenzoic, 4-hydroxybenzoic, anthranilic, cinnamic, salicylic, 4-aminosalicylic, 2-phenoxybenzoic, 2-acetoxybenzoic, and mandelic acid, sulfonic acids, such as, methanesulfonic, ethanesulfonic and β-hydroxyethanesulfonic acid.

As used herein, the term “hydrate” refers to a combination of water with a compound of formula (I) or (II) wherein the water retains its molecular state as water and is either absorbed, adsorbed or contained within a crystal lattice of the substrate molecule of formula (I) or (II).

As used herein, the term “absorbed” refers to the physical state wherein the water molecule in the hydrated, pharmaceutically acceptable acid addition salts of piperidine derivatives of the formula (I) and (II) is distributed over the surface of the solid hydrated, pharmaceutically acceptable acid addition salts of piperidine derivatives of the formula (I) and (II).

As used herein, the term “absorbed” refers to the physical state wherein the water molecule in the hydrated, pharmaceutically acceptable acid addition salts of piperidine derivatives of the formula (I) and (II) is distributed throughout the body is of the solid hydrated, pharmaceutically acceptable acid addition salts of piperidine derivatives of the formula (I) and (II).

Hydrated, pharmaceutically acceptable acid addition salts of the compounds of formula (I) and (II) are those hydrates ranging from essentially 0.10 to 5 molecules of water per molecule of substrate salt of formula (I) or (II).

As used herein, the term “azeotropic mixture” refers to a liquid mixture of two or more substances which behaves like a single substance in that the vapor produced by partial evaporation of liquid has the same composition as the liquid. The constant boiling mixture exhibits either a maximum or minimum boiling point as compared with that of other mixtures of the same substance.

As used herein, the term “azeotropic distillation” refers to a type of distillation in which a substance is added to the mixture to be separated in order to form an azeotropic mixture with one or more of the constituents of the original mixture. The azeotrope or azeotropes thus formed will have boiling points different from the boiling points of the original mixture. As used herein, the term

As used herein, the term “water-minimizing recrystallization” refers to a recrystallization wherein the ratio of anhydrous solvent to substrate hydrate is such that the percentage of water present is minimized, thereby inducing precipitation of the anhydrous form of the substrate.

As used herein, the term “aqueous recrystallization” refers to those processes wherein either 1) a solid material is dissolved in a volume of water or a water/organic solvent mixture sufficient to cause dissolution and the solid material recovered by evaporation of the solvent; 2) a solid material is treated with a minimal amount of water or a water/organic solvent mixture which is not sufficient to cause dissolution, heated to obtain dissolution and cooled to induce crystallization or 3) a solid material is dissolved in a volume of water or a water/organic solvent mixture sufficient to cause dissolution and then the solvent is partially evaporated to form a saturated solution which induces crystallization.

As used herein, the term “crystal digestion” refers to that process wherein a solid material is treated with a minimal amount of water or water/organic solvent mixture which is not sufficient to cause dissolution and either heating or stirring at ambient temperature until the desired transformation has taken place.

As used herein, the term “antisolvent” refers to a poor solvent for the substance in question which when added to a solution of the substance, causes the substance to precipitate.

As used herein, the term “suitable temperature” refers to that temperature which is sufficient to cause dissolution and to permit the precipitation of the desired substance either upon addition of an antisolvent or upon removal of the co-solvent by azeotropic distillation.

The anhydrous, pharmaceutically acceptable acid addition salts of piperidine derivatives of the formula (I) and (II) may be prepared from the corresponding hydrated, pharmaceutically acceptable acid addition salts of piperidine derivatives of the formula (I) and (II) by subjecting the corresponding hydrated, pharmaceutically acceptable acid addition salts of piperidine derivatives of the formula (I) and (II) to an azeotropic distillation.

For example, the appropriate hydrated, pharmaceutically acceptable acid addition salt of piperidine derivatives of the formula (I) and (II) is first dissolved in a volume of a suitable solvent or solvent mixture which is sufficiet to cause dissolution. Examples of such solvents are water, C₁-C₅ alkanols such as methanol, ethanol and the like; ketone solvents such as acetone, methyl ethyl ketone and the like; aliphatic ester solvents such as ethyl acetate, methyl acetate, methyl formate, ethyl formate, isopropyl acetate and the like and aqueous mixtures of these solvents, such as acetone/water, methyl ethyl ketone/water, water/acetone and water/acetone/ethyl acetate. An additional volume of the same solvent used to effect dissolution or second suitable anhydrous antisolvent is then is added to this solution, which is then heated to a boiling point which is suitable to azeotropically remove water and other low boiling components. Suitable anhydrous antisolvents for use in the azeotropic distillation are, for example, ketone solvents such as acetone, methyl ethyl ketone and the like; aliphatic ester solvents such as ethyl acetate, methyl acetate, methyl formate, ethyl formate, isopropyl acetate and the like; C₅-C₈ aliphatic solvents such as pentane, hexane and the like; aliphatic nitrites, such as acetonitrile and mixtures of these solvents such as acetone/ethyl acetate and the like. The azeotropic mixture of water and solvent is removed by distillation until the temperature changes, indicating that the azeotropic mixture is completely removed. The reaction mixture is cooled and the corresponding anhydrous, pharmaceutically acceptable acid addition salts of piperidine derivatives of the formula (I) and (II) is recovered from the reaction zone by, for example filtration.

In addition, the anhydrous, pharmaceutically acceptable acid addition salts of piperidine derivatives of the formula (I) and (II) may be prepared from the corresponding hydrated, pharmaceutically acceptable acid addition salts of piperidine derivatives of the formula (I) and (II) by subjecting the corresponding hydrated, pharmaceutically acceptable acid addition salts of piperidine derivatives of the formula (I) and (II) to a water-minimizing recrystallization.

For example, the appropriate hydrated, pharmaceutically acceptable acid addition salt of piperidine derivatives of the formula (I) and (II) is dissolved in a volume of a suitable anhydrous solvent or solvent mixture which is sufficient to cause dissolution and heated to reflux. Examples of such solvents are water, C₁-C₅ alkanols such as methanol, ethanol and the like; ketone solvents such as acetone, methyl ethyl ketone and the like; aliphatic ester solvents such as ethyl acetate, methyl acetate, methyl formate, ethyl formate, isopropyl acetate and the like and aqueous mixtures of these solvents, such as acetone/water, methyl ethyl ketone/water, water/acetone and water/acetone/ethyl acetate. An additional volume of the same solvent used to effect dissolution or second suitable anhydrous antisolvent is then added in a quantity sufficient to initiate precipitation of the anhydrous, pharmaceutically acceptable acid addition salt of piperidine derivatives of the formula (I) and (II). Suitable anhydrous antisolvents are, for example, ketone solvents such as acetone, methyl ethyl ketone and the like; aliphatic ester solvents such as ethyl acetate, methyl acetate, methyl formate, ethyl formate, isopropyl acetate and the like; mixtures of ketone solvents and aliphatic ester solvents such as acetone/ethyl acetate and the like; C₅-C₈ aliphatic solvents such as pentane, hexane and the like; aliphatic nitrites, such as acetonitrile and mixtures of these solvents such as acetone/ethyl acetate and the like as well as mixtures of water and ketone solvents such as acetone/water and the like; and mixtures of water, ketone solvents and aliphatic ester solvents such as acetone/water/ethyl acetate. The reaction mixture is cooled and the corresponding anhydrous, pharmaceutically acceptable acid addition salt of piperidine derivatives of the formula (I) and (II) is recovered from the reaction zone by, for example filtration.

Pseudomorphic forms of hydrated 4-[4-[4-(Hydroxydiphenylmethyl)-1-piperidinyl]-1-hydroxybutyl]- α,α-dimethylbenzeneacetic acid free base (Forms I′, II′, III′, IV′, V′, XIII′, and IX′) may be prepared by a variety of methods as detailed below.

### Form I′ hydrated 4-[4-[4-(hydroxydiphenylmethyl)-1-piperidinyl]-1-hydroxybutyl]-α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form I′ may be prepared from hydrated 4-[4-[4-(hydroxydiphenylmethyl)-1-piperidinyl]-1-hydroxybutyl]-α,α-dimethylbenzeneacetic acid, hydrochloride salt by dissolving the hydrated 4-[4-[4-(hydroxydiphenylmethyl)-1-piperidinyl]-1-hydroxybutyl]-α,α-dimethylbenzeneacetic acid, hydrochloride salt in a suitable organic solvent, such as methanol/water and then treating the solution with a suitable base, such as aqueous sodium bicarbonate.

### Form II′ hydrated 4-[4-[4-(hydroxydiphenylmethyl)-1-piperidinyl]-1-hydroxybutyl]-α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form II′ may be prepared from hydrated 4-[4-[4-(hydroxydiphenylmethyl)-1-piperidinyl]-1-hydroxybutyl]-α,α-dimethylbenzeneacetic acid hydrochloride salt Form II by dissolving the hydrated 4-[4-[4-(hydroxydiphenylmethyl)-1-piperidinyl]-1-hydroxybutyl]-α,α-dimethylbenzeneacetic acid hydrochloride salt Form II in a suitable organic solvent, such as boiling acetone, treating the solution with a minimum amount of water, followed by treatment of the solution with silica gel. The hydrated 4-[4-[4-(hydroxydiphenylmethyl)-1-piperidinyl]-1-hydroxybutyl]-α,α-dimethylbenzeneacetic acid Form II′ may be obtained by filtration through a suitable filter, such as 100 mesh nylon sieve.

### Form III′ hydrated 4-[4-[4-(hydroxydiphenylmethyl)-1-piperidinyl]-1-hydroxybutyl-α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form III′ may be prepared from hydrated 4-[4-[4-(hydroxydiphenylmethyl)-1-piperidinyl]-1-hydroxybutyl]-α,α-dimethylbenzeneacetic acid Form I′ by dissolving hydrated 4-[4-[4-(hydroxydiphenylmethyl)-1-piperidinyl]-1-hydroxybutyl]-α,α-dimethylbenzeneacetic acid Form I′ in a suitable organic solvent, such as methanol, stirring briefly, and then filtering through a suitable filter, such as 0.22 micrometer Millipore GVWP filter.

### Form IV′ hydrated 4-[4-[4-(hydroxydiphenylmethyl)-1-piperidinyl]-1-hydroxybutyl]-α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form IV′ may be prepared from 4-[4-[4-(hydroxydiphenylmethyl)-1-piperidinyl]-1-hydroxybutyl]-α,α-dimethylbenzeneacetic acid hydrochloride salt Form I by dissolving 4-[4-[4-(hydroxydiphenylmethyl)-1-piperidinyl]-1-hydroxybutyl]-α,α-dimethylbenzeneacetic acid hydrochloride salt Form I in water, and then adjusting the pH to approximately neutral with a suitable base, such as sodium hydroxide.

### Form V′ hydrated 4-[4-[4-(hydroxydiphenylmethyl)-1-piperidinyl]-1-hydroxybutyl]-α,α-dimethylbenzeneacetic acid

Hydrated 4-[4-[4-(hydroxydiphenylmethyl)-1-piperidinyl]-1-hydroxybutyl]-(xy,(x dimethylbenzeneacetic acid Form V′ may be prepared from 4-[4-[4-(hydroxydiphenylmethyl)-1-piperidinyl]-1-hydroxybutyl]-α,α-dimethylbenzeneacetic acid hydrochloride salt Form I by mixing 4-[4-[4-(hydroxydiphenylmethyl)-1-piperidinyl]-1-hydroxybutyl]-α,α-dimethylbenzeneacetic acid hydrochloride salt Form I with a suitable aqueous base, such as sodium hydroxide, allowing the mixture to stand at ambient temperatures for several days, then diluting with water. The hydrated 4-[-[4-(hydroxydiphenylmethyl)-1-piperidinyl]-1-hydroxybutyl]-α,α-dimethylbenzeneacetic acid Form V′ may be obtained by filtration through a suitable filter, such as Whatman GF/G.

### Form VIII′ hydrated 4-[4-[4-(hydroxydiphenylmethyl)-1-piperidinyl]-1-hydroxybutyl]-α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form VIII′ may be prepared from 4-[4-[4-(hydroxydiphenylmethyl)-1-piperidinyl]-1-hydroxybutyl]-α,α-dimethylbenzeneacetic acid hydrochloride salt form I by dissolving the 4-[4-[4-(hydroxydiphenylmethyl)-1-piperidinyl]-1-hydroxybutyl]-α,α-dimethylbenzeneacetic acid hydrochloride salt form I in a suitable organic solvent, such as methanol, adding water, then treating with a suitable base, such as aqueous sodium bicarbonate. The hydrated 4-[4-[4-(hydroxydiphenylmethyl)-1-piperidinyl]-1-hydroxybutyl]-α,α-dimethylbenzeneacetic acid Form VIII′ may be obtained by filtration.

### Form IX′ hydrated 4-[4-[4-(hydroxydiphenyimethyl)-1-piperidinyil-1-hydroxybutyl]- α,α-dimethylbenzeneacetic acid

Hydrated 4-[4-[4-(hydroxydiphenylmethyl)-1-piperidinyl]-1-hydroxybutyl]-α,α-dimethylbenzeneacetic acid Form IX′ may be prepared from 4-[4-[4-(hydroxydiphenylmethyl)-1-piperidinyl]-1-hydroxybutyl]-α,α-dimethylbenzeneacetic acid Form VIII′ by dissolving 4-[4-[4-(hydroxydiphenylmethyl)-1-piperidinyl]-1-hydroxybutyl]-α,α-dimethylbenzeneacetic acid Form VIII′ in a suitable organic solvent, such as methanol, stirring, then heating to the boiling point of the organic solvent used and then filtering the hot solution. The hydrated 4-[4-[4-(hydroxydiphenylmethyl)- 1-piperidinyl]-1-hydroxybutyl]-α,α-dimethylbenzeneacetic acid Form IX′ may be obtained by filtration.

Pseudomorphic forms of anhydrous 4-[4-[4-(Hydroxydiphenylmethyl)-1-piperidinyl]-1-hydroxybutyl]-α,α-dimethylbenzeneacetic acid free base (Form VII′) may be prepared as follows:

### Form VII′ anhydrous 4-[4-[4-(hydroxydiphenylmethyl)-1-piperidinvl]-1-hydroxybutyl]-α,α-dimethylbenzeneacetic acid

Anhydrous 4-[4-[4-(hydroxydiphenylmethyl)-1-piperidinyl]-1-hydroxybutyl]-α,α-dimethylbenzeneacetic acid Form VII′ may be prepared from hydrated 4-[4-[4-(hydroxydiphenylmethyl)-1-piperidinyl]-1-hydroxybutyl]-α,α-dimethylbenzeneacetic acid Form I′ by subjecting hydrated 4-[4-[4-(hydroxydiphenylmethyl)-1-piperidinyl]-1-hydroxybutyl]-α,α-dimethylbenzeneacetic acid Form I′ to a temperature of from about 100-200° C., preferably about 188° C. for a period of time of from about 1-48 hours, preferably about 24 hours, under an inert atmosphere.

Polymorphic forms of anhydrous 4-[4-[4-(Hydroxydiphenylmethyl)-1-piperidinyl]-1-hydroxybutyl]-α,α-dimethylbenzeneacetic acid hydrochloride (Forms I and III) may be prepared by a variety of methods as detailed below.

### Form III to Form I

For example, anhydrous 4-[4-[4-(Hydroxydiphenylmethyl)-1-piperidinyl]-1-hydroxybutyl]-α,α-dimethylbenzeneacetic acid hydrochloride (Form I) may be prepared from anhydrous 4-[4-[4-(Hydroxydiphenylmethyl)-1-piperidinyl]-1-hydroxybutyl]-α,α-dimethylbenzeneacetic acid hydrochloride (Form III), by subjecting the anhydrous 4-[4-[4-(Hydroxydiphenylmethyl)-1-piperidinyl]-1-hydroxybutyl]-α,α-dimethylbenzeneacetic acid hydrochloride (Form III) to a crystal digestion as described above.

### Form II to Form III

In addition, anhydrous 4-[4-[4-(Hydroxydiphenylmethyl)-1-piperidinyl]-1-hydroxybutyl]-α,α-dimethylbenzeneacetic acid hydrochloride (Form III) may be prepared from hydrated 4-[4-[4-(Hydroxydiphenylmethyl)-1-piperidinyl]- 1-hydroxybutyl]-α,α-dimethylbenzeneacetic acid hydrochloride (Form II), by subjecting the hydrated 4-[4-[4-(Hydroxydiphenylmethyl)-1-piperidinyl]-1-hydroxybutyl]-α,α-dimethylbenzeneacetic acid hydrochloride (Form II) to water-minimizing recrystallization as described above.

### Form II to Form I

In addition, anhydrous 4-[4-[4-(Hydroxydiphenylmethyl)-1-piperidinyl]-1-hydroxybutyl]-α,α-dimethylbenzeneacetic acid hydrochloride (Form I) may be prepared from hydrated 4-[4-[4-(Hydroxydiphenylmethyl)-1-piperidinyl]-1-hydroxybutyl]-α,α-dimethylbenzeneacetic acid hydrochloride (Form II), by subjecting the hydrated 4-[4-[4-(Hydroxydiphenylmethyl)-1-piperidinyl]-1-hydroxybutyl]-α,α-dimethylbenzeneacetic acid hydrochloride (Form II) to water-minimizing recrystallization as described above or by subjecting the hydrated 4-[4-[4-(Hydroxydiphenylmethyl)-1-piperidinyl]-1-hydroxybutyl]-α,α-dimethylbenzeneacetic acid hydrochloride (Form II) to an azeotropic distillation.

### Form IV to Form I

In addition, anhydrous 4-[4-[4-(Hydroxydiphenylmethyl)- 1-piperidinyl]-1-hydroxybutyl]-α,α-dimethylbenzeneacetic acid hydrochloride (Form I) may be prepared from hydrated 4-[4-[4-(Hydroxydiphenylmethyl)-1-piperidinyl]-1-hydroxybutyl]-α,α-dimethylbenzeneacetic acid hydrochloride (Form IV), by subjecting the hydrated 4-[4-[4-(Hydroxydiphenylmethyl)-1-piperidinyl]-1-hydroxybutyl]-α,α-dimethylbenzeneacetic acid hydrochloride (Form IV) to water-minimizing recrystallization or to an azeotropic distillation as described above.

The hydrated, pharmaceutically acceptable acid addition salts of piperidine derivatives of the formula (I) may be prepared from the corresponding compound of the formula (II) wherein R₃ is -COOalkyl by subjecting the corresponding compound of the formula (II) wherein R₃ is -COOalkyl to a reduction using an appropriate reducing agent, such as sodium borohyride, potassium borohydride, sodium cyanoborohydride, or tetramethylammonium borohydride in a suitable solvent, such as, methanol, ethanol, isopropyl alcohol or n-butanol, aqeuous mixtures thereof or basic solutions thereof, at temperatures ranging from about 0° C. to the reflux temperature of the solvent, and the reaction time varies from about ½ hour to 8 hours. After quenching and acidifying with an suitable acid, such as hydrochloric acid, the hydrated, pharmaceutically acceptable acid addition salts of piperidine derivatives of the formula (I) are recovered from the reaction zone by crystallization and filtration.

In addition, the hydrated, pharmaceutically acceptable acid addition salts of piperidine derivatives of the formula (I) and (II) may be prepared from the corresponding anhydrous, pharmaceutically acceptable acid addition salts of the formula (I) and (II) by subjecting the corresponding anhydrous, pharmaceutically acceptable acid addition salts of formula (I) and (II) to an aqueous recrystallization.

For example, the appropriate anhydrous, pharmaceutically acceptable acid addition salt of piperidine derivatives of the formula (I) and (II) is treated with a minimal volume of water or suitable water/organic solvent mixture which is insufficient to cause dissolution and heated to reflux. The reaction mixture is cooled and the corresponding hydrated, pharmaceutically acceptable acid addition salt of piperidine derivatives of the formula (I) and (II) is recovered from the reaction zone by, for example filtration. Alternatively, the appropriate anhydrous, pharmaceutically acceptable acid addition salt of piperidine derivatives of the formula (I) and (II) is treated with a volume of water or a suitable water/organic solvent mixture which is sufficient to cause dissolution and the water or water/organic solvent is partially or completely evaporated to a volume which induces crystallization of the hydrated, pharmaceutically acceptable acid addition salts of piperidine derivatives of the formula (I) and (II). Suitable solvents for use in the above recrystallization are water, acetone/water, ethanol/water, methyl ethyl ketone/aqueous methanol, methyl ethyl ketone/water and the like.

The pseudomorphic forms of hydrated 4-[4-[4-(Hydroxydiphenylmethyl)-1-piperidinyl]-1-hydroxybutyl]-α,α-dimethylbenzeneacetic acid hydrochloride (Forms II and IV) may be prepared by a variety of methods as detailed below.

### Ethyl Ester/Ketone to Form II

Hydrated 4-[4-[4-(Hydroxydiphenylmethyl)-1-piperidinyl]-1-hydroxybutyl]-α,α-dimethylbenzeneacetic acid hydrochloride (Form IV) may be prepared from ethyl 4-[4-[4-(hydroxydiphenylmethyl)-1-piperidinyl]-1-oxobutyl]-α,α-dimethylbenzeneacetate, hydrochloride or free base as described above for the general preparation of the hydrated, pharmaceutically acceptable acid addition salts of piperidine derivatives of the formula (I) from the corresponding compound of the formula (II) wherein R₃ is -COOalkyl, but rapdily adding water over a period of time ranging from 1 minute to 45 minutes at a temperature range of about -20° C. to 50° C. to precipitate the hydrated 4-[4-[4-(Hydroxydiphenylmethyl)-1-piperidinyl]-1-hydroxybutyl]-α,α-dimethylbenzeneacetic acid hydrochloride (Form II).

### Ethyl Ester/Ketone to Form IV

Hydrated 4-[4-[4-(Hydroxydiphenylmethyl)-1-piperidinyl]-1-hydroxybutyl]-α,α-dimethylbenzeneacetic acid hydrochloride (Form IV) may be prepared from ethyl 4-[4-[4-(hydroxydiphenylmethyl)-1-piperidinyl]-1-oxobutyl]-α,α-dimethylbenzeneacetate, hydrochloride or free base as described above for the general preparation of the hydrated, pharmaceutically acceptable acid addition salts of piperidine derivatives of the formula (I) from the corresponding compound of the formula (II) wherein R₃ is -COOalkyl, but slowly adding water over a period of time ranging from about 30 minutes to 24 hours and at a temperature range of about 0° C. to 50° C., optionally with seeding, to precipitate the hydrated 4-[4-[4-(Hydroxydiphenylmethyl)-1-piperidinyl]-1-hydroxybutyl]-α,α-dimethylbenzeneacetic acid hydrochloride (Form IV).

### Form I to Form II

Hydrated 4-[4-[4-(Hydroxydiphenylmethyl)-1-piperidinyl]-1-hydroxybutyl]-α,α-dimethylbenzeneacetic acid hydrochloride (Form II) may be prepared from anhydrous 4-[4-[4-(Hydroxydiphenylmethyl)-1-piperidinyl]-1-hydroxybutyl]-α,α-dimethylbenzeneacetic acid hydrochloride (Form I) by subjecting hydrated 4-[4-[4-(Hydroxydiphenylmethyl)-1-piperidinyl]-1-hydroxybutyl]-α,α-dimethylbenzeneacetic acid hydrochloride (Form II) to an aqueous recrystallization as defined above.

### Preferred Solution Conformations

Form I′″, Form II′″, Form III′″, Form IV′″, Form V′″, Form VII′″, Form VIII′″, Form IX′″ Form I″, Form II″, Form III″ and Form IV″ preferred aqueous solution conformations may all be prepared by dissolving the appropriate corresponding solid pseudomorphic or polymorphic, anhydrous or hydrated 4-[4-[4-(hydroxydiphenylmethyl)-1-piperidinyl]-1-hydroxybutyl]-α,α-dimethylbenzeneacetic acid free base or hydrochloride salt compound (i.e., Form I′, Form II′, Form III′, Form IV′, Form V′, Form VII′, Form VII′, Form IX′ Form I, Form II, Form III and Form IV) in a non-buffered aqueous solution, pH of 0.5-13. The solution may be allowed to stand for a period of time, preferably 1-20 days, more preferably 5-15 days, and may optionally be stirred or vibrated during that period.

Alternatively, Form I′″, Form II′″, Form III′″, Form IV′″, Form V′″, Form VII′″, Form VIII′″, Form IX′″ Form I″, Form II″, Form III″ and Form IV″ preferred aqueous solution conformations may be prepared by orally administering the appropriate corresponding solid pseudomorphic or polymorphic, anhydrous or hydrated 4-[4-[4-(hydroxydiphenylmethyl)-1-piperidinyl]-1-hydroxybutyl]-α,α-dimethylbenzeneacetic acid free base or hydrochloride salt compound (i.e., Form I′, Form II′, Form III′, Form IV′, Form V′, Form VII′, Form VIII′, Form IX′ Form I, Form II, Form III and Form IV) compound to human subjects.

Starting materials for use in the present invention are readily available to one of ordinary skill in the art. For example, ethyl 4-[4-[4-(Hydroxydiphenylmethyl)-1-piperidinyl]-1-oxobutyl]-α,α-dimethylbenzeneacetate, hydrochloride is described in U.S. Pat. No. 4,254,129, Mar. 3, 1981.

The following examples present typical processes for preparing the anhydrous and hydrated, pharmaceutically acceptable acid addition salts of piperidine derivatives of the formula (I) and (II), polymorphs and pseudomorphs therof. These examples are understood to be illustrative only and are not intended to limit the scope of the present invention in any way. As used herein, the following terms have the indicated meanings: “g” refers to grams; “mol” refers to mole; “mmol” refers to millimoles; “mL” refers to milliliters; “bp” refers to boiling point; “mp” refers to melting point; “°C” refers to degrees Celsius; “mm Hg” refers to millimeters of mercury; “μL” refers to microliters; “μg” refers to micrograms; and “μM” refers to micromolar.

Differential Scanning Calorimetry analysis on the free base Forms I′, II′, III′, IV, V′, VII′, VIII′ and IX′ were performed using a Perkin-Elmer DSC 7 with open aluminum pans. Calibration of the DSC 7 was verified prior to sample analysis using an indium standard The samples were heated to 250° C. at 10° C./minute with a 22 mL/minute nitrogen purge.

Differential Scanning Calorimetry analysis on the hydrochloride salt Forms I, II, III and IV were performed using a TA 2910 DSC with open aluminum pans. The samples were heated to 240° C. at 5° C./minute with a 50 mL/minute nitrogen purge.

X-Ray Powder Diffraction analyses on the free base Forms I′, II′, III′, IV, V′, VII′, VIII′ and IX′ were performed as follows:

The samples were loaded into a platinum sample holder for the XRPD pattern measurement. The XRPD patterns were measured using a powder diffractometer equipped with a Cu X-ray tube source, primary beam monochromator, and position sensitive detector (PSD). Source slits of 2 and 4 mm, and detector slits of 0.5 and 0.3 mm were used for data collection. The source was operated at 45 kV and 40 mA, using a Kevex PsiPeltier-cooled silican detector and the sample was illuminated with Cu Kα₁ radiation. XRPD data were collected from 2 to 42° 2θ at a rate of 1.0°2θ/minute. Calibration of the XDS 2000 was verified using the 100% line of platinum metal.

Peak positions and intensities for the most prominent features were measured using a double-derivative peak picking method. Ten to fifteen X-ray peaks which exhibited the strongest intensity were reported. The intensities are rounded to the nearest 1%. Certain peaks appear sensitive to preferred orientation that is caused by changes in crystallite morphology. This can result in large changes in the I/I_{°}value.

X-Ray Powder Diffraction analyses on the hydrochloride salt Forms I, II, III and IV were performed as follows:

The samples were loaded into a quartz (zero scatter) sample holder for the XRPD pattern measurement. The XRPD patterns were measured using a powder diffractometer equipped with a Co X-ray tube source, primary beam monochromator, and position sensitive detector (PSD). The incident beam was collimated using a 1° divergence slit. The active area on the PSD subtended approximately 5°2θ. The source was operated at 35 kV and 30 mA and the sample was illuminated with Co Kα₁ radiation. XRPD data were collected from 5 to 55° 2θ at a rate of 0.25°2θ minute and a step width of 0.02°2θ. The XRPD patterns were measured without the addition of an internal calibrant.

Peak positions and intensities for the most prominent features were measured using a double-derivative peak picking method. X-ray peaks with I/I_{°}greater than 20% were reported. The cutoff was chosen arbitrarily. The intensities are rounded to the nearest 5%. Certain peaks appear sensitive to preferred orientation that is caused by changes in crystallite morphology. This results in large changes in the I/I_{°}value.

### Example 1 -Preparation of Form II

4-[4-[4-(Hydroxydiphenylmethyl)-1-piperidinyl]-1-hydroxybutyl]- α,α-dimethylbenzeneacetic acid hydrochloride

Method A

Mix ethyl 4-[4-[4-(hydroxydiphenylmethyl)-1-piperidinyl]-1-oxobutyl]-α,α-dimethylbenzeneacetate, hydrochloride (101.92 g, 0.1807 mol) and methanol (510 mL) and stir. Rapidly add 50% sodium hydroxide (72.27 g, 0.903 mol) and wash in with water (61 mL). Heat to reflux for 2 hours, allow to cool to 35° C. and treat with sodium borohydride (3.42 g, 0.0903 mol). Add water (100 mL) and maintain at 35° C. for 10 hours. Add 37% hydrochloric acid (53.0 g) to adjust pH to 11.5. Add acetone (26.5 mL) and water (102 mL). Hold at 35° C. for 2 hours and adjust to pH 2.5 with 37% hydrochloric acid (44.69g). Dilute with water (408 mL), cool to −15° C., stir for 1.5 hours and collect the precipitate by vacuum filtration.

Wash the filtercake with deionized water (3×100 mL) and vacuum dry to give 4-[4-[4-(hydroxydiphenylmethyl)-1-piperidinyl]-1-hydroxybutyl]-α,α-dimethylbenzeneacetic acid hydrochloride hydrate (97.10 g).

Method B

Place ethyl 4-[4-[4-(hydroxydiphenylmethyl)-1-piperidinyl]-1-oxobutyl]-α,α-dimethylbenzeneacetate, hydrochloride (60.01 g, 0.106 mol) in a 1-L three necked round-bottom flask and fit the flask with a mechanical stirrer, a Claisen head, a thermometer and a reflux condenser with a nitrogen bubbler on top. Add methanol (300 mL) and turn the stirrer on. Dilute the slurry with water (60 mL) and heat to 52-54° C. over 15-20 minutes. Hold at 52° C. for 2 hours and then add 50% is sodium hydroxide (42.54 g, 0.532 mol). Heat at 73° C. for approximately 1 hour, 45 minutes, cool to less than 35° C. using a water bath and then add sodium borohydride (2.02 g, 0.0534 mol). Stir overnight at 35° C., treat with acetone (15.5 mL) and stir for 2 hours at 35° C. Acidify the mixture to a pH of 1.85 with 28% hydrochloric acid (75.72 g), dilute with water (282 mL), stir for about 30 minutes and cool over about 2 hours to −15° C. Filter the solids off and wash with water (2×75 mL) and ethyl acetate (2×75 mL). Vacuum dry the solid and allow to stand for 2 days to give 4-[4-[4-(hydroxydiphenylmethyl)-1-piperidinyl]-1-hydroxybutyl]-α,α-dimethylbenzeneacetic acid hydrochloride hydrate (Form II) (57.97 g, 91.5%) as a fine powder.

Method C

Place ethyl 4-[4-[4-(hydroxydiphenylmethyl)-1-piperidinyl]-1-oxobutyll-α,α-dimethylbenzeneacetate (56.12 g, 0.1064 mol) in a 1-L three necked round-bottom flask and fit the flask with a mechanical stirrer, a Claisen head, a thermometer and a reflux condenser with a nitrogen bubbler on top. Add methanol (300 mL) and turn the stirrer on. Dilute the slurry with water (60 mL) and heat to reflux using a heating mantle controlled by a Therm-O-Watch. When the mixture reaches about 35° C., treat with 50% sodium hydroxide (34.05 g, 0.4256 mol) and rinse in with water (42 mL). Stir at reflux for 2 hours, 15 minutes, cool over 1 hour to 35° C. and then treat with sodium borohydride (2.02 g, 0.0534 mol). Stir for 7.5 hours and allow to stand at room temperature without stirring for 1.75 days. Warm the mixture to 35° C. and quench with acetone (1 5.5 mL, 0.21 mol) and stir for 2 hours. Add water (60 mL) and adjust the pH to 2.5 with 32% hydrochloric acid (65.22 g). Cool to 40° C. and rinse the pH probe with water (25 mL). Add water over about 30 minutes (192 mL), hold the temperature at 33° C. for 10 minutes and add a few seed crystals. Cool the slurry to −12° C. over about 45 minutes and isolate the solid by filtration (586.2 g). Wash with water (2×100 mL) and then with ethyl acetate (100 mL, prechilled to about −10° C.). Vacuum dry overnight (1 mmHg, 50° C.) to give 4-[4-[4-(hydroxydiphenylmethyl)-1-piperidinyl]-1-hydroxybutyl]-α,α-dimethylbenzeneacetic acid hydrochloride hydrate (Form II) (58.86 g, 98%) as a white solid.

### Example 2-Preparation of Form IV

### 4-[4-[4-(hydroxydiphenylmethyl)-1-piperidinyl]-1-hydroxybutyl]-α,α-dimethylbenzeneacetic acid hydrochloride (Form IV)

Place ethyl 4-[4-[4-(hydroxydiphenylmethyl)-1-piperidinyl]-1-oxobutyl]-α,α-dimethylbenzeneacetate (56.12 g, 0.1064 mol) in a 1-L three necked round-bottom flask and fit the flask with a mechanical stirrer, a Claisen head, a thermometer and a reflux condenser with a nitrogen bubbler on top. Add methanol (300 mL) and turn the stirrer on. Dilute the slurry with water (60 mL) and heat to reflux using a heating mantle controlled by a Therm-O-Watch. When the mixture reaches about 35° C., treat with 50% sodium hydroxide (34.05 g, 0.4256 mol) and rinse in with water (42 mL). Stir at reflux for 2 hours, 15 minutes, cool over 1 hour to 35° C. and then treat with sodium borohydride (2.02 g, 0.0534 mol). Stir for 7.5 hours and allow to stand at room temperature without stirring for 1.75 days. Warm the mixture to 35° C. and quench with acetone (15.5 mL, 0.21 mol) and stir for 2 hours. Add water (60 mL) and adjust the pH to 2.5 with 32% hydrochloric acid (65.22 g). Cool to 40° C. and rinse the pH probe with water (25 mL). Hold the temperature at 33° C. for 10 minutes, add a few seed crystals and add water over about 4 hours (192 mL) at 35° C. Cool the slurry to −12° C. over about 45 minutes and isolate the solid by filtration (586.2 g). Wash with water (2×100 mL) and then with ethyl acetate (100 mL, prechilled to about −10° C.). Vacuum dry overnight (1 mmHg, 50° C.) to give 4-[4-[4-(hydroxydiphenylmethyl)-1-piperidinyl]-1-hydroxybutyl]-α,α-dimethylbenzeneacetic acid hydrochloride hydrate (Form IV); mp 115-116° C. (dec).

TABLE 13

| D-Space, Angstroms | Intensity, I/Iₒ, % |
|---|---|
| 10.3 | 60 |
| 6.97 | 45 |
| 6.41 | 50 |
| 5.55 | 30 |
| 5.32 | 100 |
| 5.23 | 55 |
| 5.11 | 35 |
| 4.98 | 25 |
| 4.64 | 30 |
| 4.32 | 35 |
| 4.28 | 75 |
| 4.12 | 50 |
| 4.02 | 45 |
| 3.83 | 60 |
| 3.65 | 20 |
| 3.51 | 55 |
| 3.46 | 25 |
| 2.83 | 20 |

### Example 3-Conversion of Form II to Form I

4-[4-[4-(hydroxydiphenylmethyl)-1-piperidinyl]-1-hydroxybutyl]-α,α-dimethylbenzeneacetic acid hydrochloride (Form I) Treat 4-[4-[4-(hydroxydiphenylmethyl)-1-piperidinyl]-1-hydroxybutyl]-α,α-dimethylbenzeneacetic acid hydrochloride hydrate (Form II) (20.0 g, 0.0355 mol) with deionized water (2 g) and add acetone (60 mL) in small portions over several minutes with stirring. Filter through filter aid and wash the filter cake with acetone (30 mL). Wash the filtercake with acetone (22 mL), reflux filtrate and then slowly add ethyl acetate (32 mL over 15 minutes) keeping the mixture at reflux. Reflux for 10 minutes, then slowly add additional ethyl acetate (23 mL over 10 minutes) and reflux for an additional 15 minutes. Add additional ethyl acetate (60 mL over 5-10 minutes) and continue refluxing for 15 minutes. Cool to approximately 8° C. in an ice bath, filter the solid and wash with ethyl acetate (85 mL). Vacuum dry at 55° C. for 1.5 hours to give the title compound (18.16 g, 95%).

### Example 4-Conversion of Form II to Form I

### 4-[4-[4-(Hydroxydiphenylmethyl)-1-piperidinyl]-1-hydroxybutyl]-α,α-dimethylbenzeneacetic acid hydrochloride

Method A:

Treat 4-[4-[4-(hydroxydiphenylmethyl)-1-piperidinyl]-1-hydroxybutyl]-α,α-dimethylbenzeneacetic acid hydrochloride hydrate (Form II) (5.00 g, 0.0083 mol) with methylethyl ketone (130 mL). Slowly add water (0.4 mL), filter through filter aid and wash the filter cake with methylethyl ketone (20 mL). Heat to reflux and distill off 75 mL of solvent, cool to −15° C. and collect by vacuum filtration. Wash with methylethyl ketone (2×10 mL) and vacuum dry at 60° C. to give the title compound (4.33 g, 97%); mp 196-198° C.

Method B:

Treat 4-[4-[4-(hydroxydiphenylmethyl)-1-piperidinyl]-1-hydroxybutyl]-α, α-dimethylbenzeneacetic acid hydrochloride hydrate (Form II) (1.4 g) with acetone (60 mL) and heat to reflux. Reduce the volume to approximately 35 mL to remove all water which boils off as an azeotrope (88/12:acetone/water). Cool the solution and collect the title compound as a crystalline solid.

Method C:

Mix 4-[4-[4-(hydroxydiphenylmethyl)-1-piperidinyl]-1-hydroxybutyl]-α,α-dimethylbenzeneacetic acid hydrochloride hydrate (Form II) (53.88 g, 0.100 mol) and add water (4.79 g) and methyl ethyl ketone (240 mL). Stir until the solid is slurried up and add additional methyl ethyl ketone (1 L). Stir for 0.5 hours, filter through a pad of filter aid, wash the filtercake with methyl ethyl ketone (100 mL) and transfer the filtrate and wash to a 2L, 3-necked flask fitted with a thermometer, mechanical stirrer and distillation head. Distill off a total of 721 mL of methyl ethyl ketone, cool and stir over 1 hour to 40° C. Cool to −15° C. and hold for 10 minutes. Collect the solid by vacuum filtration and wash the filtercake with methyl ethyl ketone (2×65 mL) and vacuum dry at 55° C. overnight to give the title compound is (52.76 g, 97.9%); mp 197.5-200° C.

Method D:

Treat 4-[4-[4-(hydroxydiphenylmethyl)-1-piperidinyl]-1-hydroxybutyl]-α, α-dimethylbenzeneacetic acid hydrochloride hydrate (Form II) (40.0 g, 0.0696 mol, assayed at 93.6% purity, having 0.89 g water present and 35.1 g, 0.0575 mol, assayed at 88.0% purity, having 2.47 g water present) with water (8.30 g; the amount calculated to bring the weight of water present to 17% of the anhydrous weight of 4-[4-[4-(hydroxydiphenylmethyl)-1-piperidinyl]-1-hydroxybutyl]- α,α-dimethylbenzeneacetic acid hydrochloride hydrate, taking into account the water in the hydrated salt). Add methyl ethyl ketone (approximately 500 mL) and stir until most of the solids dissolve. Add additional methyl ethyl ketone (700 mL) in portions over approximately 10 minutes and continue stirring for ½ hour. Filter through a thin pad of filter aid, wash the filtercake and flask with additional methyl ethyl ketone (100 mL) and transfer to a boiling flask fitted with a thermometer, mechanical stirrer, heating mantle, a 12-plate Oldershaw (vacuum-jacketed) distillation column and a distillation head with the capability of regulating the reflux ratio in a rough fashion, washing in with additional methyl ethyl ketone (100 mL). Distill off 450 mL of solvent, cool to −15° C. and filter the solid. Wash with methyl ethyl ketone (2×100 mL) and dry to give the title compound (68.3 g, 99.9%); mp 197-199° C.

Method E

Bring methyl ethyl ketone (4 mL) to a boil and add 4-[4-[4-(hydroxydiphenylmethyl)-1-piperidinyl]-1-hydroxybutyl]-α,α-dimethylbenzeneacetic acid hydrochloride (500 mg). Decant the top layer and add methyl ethyl ketone (3 mL) to the aqeuous layer. Boil the solution until the temperature reached 79° C., reduce the volume by 25%, remove from heat and cover with aluminum foil. Allow the solution to cool, filter the resulting crystals and air dry to give the title compound.

### Example 5-Conversion of Form I to Form II

4-[4-[4-(Hydroxydirhenylmethyl)-1-piperidinyl]-1-hydroxybutyl]-α, α-dimethylbenzeneacetic acid hydrochloride hydrate

Method A

Treat 4-[4-[4-(hydroxydiphenylmethyl)-1-piperidinyl]-1-hydroxybutyl]-α, α-dimethylbenzeneacetic acid hydrochloride (Form I) (2.0 g) with ethanol (4 mL) and deionized water (20 mL). Heat at 80° C. until a solution is formed and then stir at room temperature for 23 hours. Filter the resulting slurry, wash with water (2×10 mL) and dry under vacuum at 35° C. overnight to give the title compound (1.88 g); mp 100-105° C.

TABLE 14

| D-Space, Angstroms | Intensity, I/Iₒ, % |
|---|---|
| 11.41 | 20 |
| 7.98 | 20 |
| 7.83 | 45 |
| 6.58 | 45 |
| 6.42 | 60 |
| 5.66 | 20 |
| 5.52 | 45 |
| 5.39 | 30 |
| 5.23 | 65 |
| 5.14 | 45 |
| 4.86 | 65 |
| 4.72 | 100 |
| 4.45 | 65 |
| 4.40 | 45 |
| 4.32 | 45 |
| 4.18 | 45 |
| 4.06 | 65 |
| 4.02 | 55 |
| 3.85 | 25 |
| 3.79 | 75 |
| 3.74 | 95 |
| 3.61 | 80 |
| 3.56 | 25 |
| 3.47 | 65 |
| 3.41 | 20 |
| 2.74 | 20 |

Method B

Mix water (35.5 mL), methanol (26.3 mL) and sodium chloride (2.59 g). Add 4-[4-[4-(hydroxydiphenylmethyl)-1-piperidinyl]-1-hydroxybutyl]-α,α-dimethylbenzeneacetic acid hydrochloride (Form I) (4.77 g). Heat to reflux on a steam bath until dissolution and cool to -−10° C. Filter the resulting solid, wash with water (2×25 mL) and vacuum dry overnight to give the title compound (4.80 g).

### Example 6-Conversion of Form II into Form III

4-[4-[4-(Hydroxydiphenylmethyl)-1-piperidinyl]-1-hydroxybutyl]-α,α-dimethylbenzeneacetic acid hydrochloride (Form III)

Place 4-[4-[4-(Hydroxydiphenylmethyl)-1-piperidinyl]-1-hydroxybutyl]-α,α-dimethylbenzeneacetic acid hydrochloride hydrate (Form II) (55.56 g, 0.0929 mol having 10% water) in a pressure bottle along with water (2.96 g) and acetone (38.1 g). Seal the bottle tightly and heat to approximately 80° C. Cool to about 50° C., filter through filter aid in a coarse sintered glass funnel and dilute with acetone (90 g). Transfer to a 1 L flask fitted with a mechanical stirrer, thermometer and a reflux condenser. Heat the mixture to reflux and allow to cool and stir over the weekend. Cool to −15° C. and filter on a coarse sintered glass funnel, wash with ethyl acetate (2×50 mL) and vacuum dry at 50° C.

Place a majority of the solid obtained (45.24 g) in a 500 mL three necked flask fitted with a mechanical stirrer, thermometer and a reflux condenser. Add acetone (240 mL) and water (4.82 g) and reflux the mixture overnight. Allow the slurry to cool to 35° C. and place in an ice water bath and cool to less then 5° C. Filter the solid off on a coarse sintered glass funnel, wash with ethyl acetate (50 mL) and vacuum dry at 5° C. for several hours to give the title compound as a white crystalline powder (43.83 g, 97%); mp 166.5-170.5° C.

TABLE 15

| D-Space, Angstroms | Intensity, I/Iₒ, % |
|---|---|
| 8.95 | 95 |
| 4.99 | 20 |
| 4.88 | 100 |
| 4.75 | 35 |
| 4.57 | 25 |
| 4.47 | 25 |
| 4.46 | 20 |
| 3.67 | 20 |
| 3.65 | 25 |

### Example 7-Conversion of Form III into Form I

4-[4-[4-(Hydroxydiphenyimethyl)-1-piperidinyl]-1-hydroxybutyl]-α,α-dimethylbenzeneacetic acid hydrochloride (Form I)

Place 4-[4-[4-(Hydroxydiphenylmethyl)-1-piperidinyl]-1-hydroxybutyl]-α, α-dimethylbenzeneacetic acid hydrochloride (Form III) (40.0 g as an ethyl acetate wetcake −27.9 g dry basis) in a 1 L three necked flask fitted with a mechanical stirrer, thermometer and a reflux condenser. Add acetone (240 mL) and heat the mixture to reflux for about 20 hours. Cool the slurry to −15° C. and isolate the solids by filtration on a coarse sintered glass frit funnel. Wash with ethyl acetate (50 mL) and vacuum dry overnight to give the title compound (26.1 g, 93.7%); mp 197.5-199.5° C.

TABLE 16

| D-Space, Angstroms | Intensity, I/Iₒ, % |
|---|---|
| 11.75 | 35 |
| 7.23 | 35 |
| 6.24 | 60 |
| 5.89 | 40 |
| 5.02 | 20 |
| 4.94 | 30 |
| 4.83 | 100 |
| 4.44 | 30 |
| 3.93 | 75 |
| 3.83 | 20 |
| 3.77 | 85 |
| 3.71 | 25 |
| 3.62 | 30 |
| 3.32 | 25 |
| 3.31 | 20 |

### Example 8-Conversion of Form IV into Form I

4-[4-[4-(Hydroxydiphenyimethyl)-1-piperidinyl]-1-hydroxubutyl]-α,α-dimethylbenzeneacetic acid hydrochloride (Form I)

Place 4-[4-[4-(hydroxydiphenylmethyl)-1-piperidinyl]-1-hydroxybutyl]-α,α-dimethylbenzeneacetic acid hydrochloride hydrate (Form IV) (54.35 g, 0.0970 mol, having 4% water present) in a pressure bottle along with water (4.16 g) and acetone (38.1 g). Seal the bottle tightly and heat to approximately 80° C. Cool to less then 60° C., filter through filter aid in a coarse sintered glass funnel and rinse the filter cake with acetone (32.4 g). Place acetone (215 g) in a 1 L three necked flask fitted with a mechanical stirrer, thermometer, a reflux condenser and containing a small amount of Form I crystals and heat to reflux. Add a portion of the acetone/water solution of 4-[4-[4-(hydroxydiphenylmethyl)-1-piperidinyl]-1-hydroxybutyl]-α,α-dimethylbenzeneacetic acid hydrochloride hydrate (Form IV) (47.65 g) to the refluxing acetone over about 10 minutes. Slowly add ethyl acetate (1 57.5 g) over 45 minutes then add the remaining portion of the acetone/water solution of 4-[4-[4-(hydroxydiphenylmethyl)-1-piperidinyl]-1-hydroxybutyl]-α,α-dimethylbenzeneacetic acid hydrochloride hydrate (Form IV), rinsed in with about 20 mL of acetone. Add additional ethyl acetate (157.5 g) over 45 minutes to 1 hour, maintaining the slurry at reflux. Stir for 15 minutes, cool to −15° C. and vacuum filter the white solid on a 350 mL coarse sintered glass funnel. Wash the solids with ethyl acetate (2×50 mL) and vacuum dry overnight to give the title compound (50.36 g, 97%); mp 198-199.5° C.

TABLE 17

| D-Space, Angstroms | Intensity, I/Iₒ, % |
|---|---|
| 14.89 | 20 |
| 11.85 | 20 |
| 7.30 | 20 |
| 6.28 | 70 |
| 5.91 | 25 |
| 5.55 | 20 |
| 5.05 | 25 |
| 4.96 | 55 |
| 4.85 | 100 |
| 4.57 | 45 |
| 4.45 | 55 |
| 3.94 | 45 |
| 3.89 | 20 |
| 3.84 | 20 |
| 3.78 | 60 |
| 3.72 | 35 |
| 3.63 | 20 |
| 3.07 | 20 |
| 3.04 | 20 |
| 2.45 | 20 |

### Example 9: Preparation of hydrated 4-[4-[4-(hydroxydiphenylmethyl)-1-piperidinyl-1-hydroxybutyl]-α,α-dimethylbenzeneacetic acid Form I′

Dissolve 4-[4-[4-(hydroxydiphenylmethyl)-1-piperidinyl]-1-hydroxybutyl]-α,α-dimethylbenzeneacetic acid hydrochloride salt hydrate (23 g) in MeOH (200 mL). Dilute to 300 mL with water and add a solution of 3.24 g (0.0387 mole) NaHCO3 in 25 mL water. Allow to stand for 5 minutes and filter. Wash with water and dry at 58° C. for 7 hours, then let stand overnight. Example 10: Preparation of hydrated 4-[4-[4-(hydroxydiphenylmethyl)-1-piperidinyl]-1-hydroxybutyl]-α,α-dimethylbenzeneacetic acid Form II′

Boil reagent grade acetone (40 mL) and add approximately 500 mg 4-[4-[4-(hydroxydiphenylmethyl)-1-piperidinyl]-1-hydroxybutyl]-α,α-dimethylbenzeneacetic acid hydrochloride salt Form II. As the solution boils, add approximately 10 drops of water until the solution becomes clear. Pour the hot solution into 50 mL of 200 mesh silica gel (Aldrich lot # 04806MF) and seal with a glass stopper. Allow the solution to stand undisturbed for 5 days. Harvest the crystals using a 100 mesh nylon sieve.

### Example 11: Preparation of hydrated 4-[4-[4-(hydroxydiphenylmethyl)-1-piperidinyl]-1-hydroxybutyl1-α,α-dimethylbenzeneacetic acid Form III′

Dissolve approximately 150 mg of 4-[4-[4-(hydroxydiphenylmethyl)-1-piperidinyl]-1-hydroxybutyl]-α,α-dimethylbenzeneacetic acid Form I′ to 400 mL of MeOH. Stir for approximately 30 seconds and filter the precipitate through a 0.22 micrometer Millipore GVWP filter and dry at 34+ C. overnight.

### Example 12: Preparation of hydrated 4-[4-[4-(hydroxydiphenylmethyl)-1-piperidinyl]-1-hydroxybutyl1-α,α-dimethylbenzeneacetic acid Form IV′

Dissolve 315 mg 4-[4-[4-(hydroxydiphenylmethyl)-1-piperidinyl]-1-hydroxybutyl]-α,α-dimethylbenzeneacetic acid hydrochloride salt Form I in 100 mL of deionized water. Stir gently for 45 minutes at room temperature for 45 minutes. Filter through a 0.45 micrometer PTFE filter, adjust the pH to 7.54 with 0.1 M NaOH, filter the precipitate and dry overnight at 35° C.

### Example 13: Preparation of hydrated 4-[4-[4-(hydroxydiphenylmethyl)-1-piperidinyl]-1-hydroxybutyl]-α,α-dimethylbenzeneacetic acid Form V′

Mix 250 mg 4-[4-[4-(hydroxydiphenylmethyl)-1-piperidinyl]-1-hydroxybutyl]-α,α-dimethylbenzeneacetic acid hydrochloride salt Form I with 20 mL 0.227 M NaOH in an ampule. Seal the ampule and place in a 25° C. constant temperature bath. After 6 days, add 3 mL of deionized water and stir for 10 minutes. Filter through a Whatman GF/G filter and dry at 35° C. overnight.

### Example 14: Preparation of hydrated 4-[4-[4-(hydroxydiphenylmethyl)-1-piperidinyl-1-hydroxybutyl]-α,α-dimethylbenzeneacetic acid Form VII′

Dissolve approximately 50 grams of 4-[4-[4-(hydroxydiphenylmethyl)-1-piperidinyl]-1-hydroxybutyl]-α,α-dimethylbenzeneacetic acid hydrochloride salt form I in 400 mL of MeOH. Add 600 mL of Nanopure water and stir for 15 minutes. Add 50 mL of a saturated solution of sodium bicarbonate. Stir an additional 30 minutes at room temperature, collect the solid and wash with water. Dry overnight at 80° C.

### Example 15: Preparation of hydrated 4-[4-[4-(hydroxydiphenylmethyl)-1-piperidinyl]-1-hydroxybutyl]-α,α-dimethylbenzeneacetic acid Form IX′

Dissolve approximately 14 g of 4-[4-[4-(hydroxydiphenylmethyl)-1-piperidinyl]-1-hydroxybutyl]-α,α-dimethylbenzeneacetic acid Form VII′ in 1.2 L of MeOH. Stir at room temperature for 1 hour, then heat to 60° C. for 30 minutes. Filter the solution hot and reduce in volume to 800 mL. Cool to room temperature, filter and wash with 50 mL MeOH. Dry at 80° C.

### Example 16: Preparation of anhydrous 4-[4-[4-(hydroxydiphenylmethyl)-1-piperidinyl]-1-hydroxybutyl]-α,α-dimethylbenzeneacetic acid Form VII′

Place approximately 2 g of 4-[4-[4-(hydroxydiphenylmethyl)-1-piperidinyl]-1-hydroxybutyl]-α,α-dimethylbenzeneacetic acid Form I′ into an oven and heat to approximately 188° C. Purge the oven with nitrogen gas and maintain at 188° C. for approximately 24 hours.

### Example 17: Preparation/Identification of Preferred solution conformation Form I′″4-[4-[4-(hydroxydiphenylmethyl)-1-piperidinyl]-1-hydroxybutyl]-α,α-dimethylbenzeneacetic acid

Into each of fourteen to twenty-three 10-mL ampules (Wheaton Flint Glass), approximately 50.0 mg of Form I′ hydrated 4-[4-[4-(hydroxydiphenylmethyl)-1-piperidinyl]-1-hydroxybutyl]-α,α-dimethylbenzeneacetic acid free base were weighed. To these ampules various concentrations of acid (HCI) or base (NaOH) were added ranging in concentration from 0 to 0.2 M with the total volume in each ampule being 10 mL. The ampules were then heat sealed (Cozzoli Model HS-1), placed into a 25° C. constant temperature bath controlled to within 0.01° C. (Tamson TEV-45) and attached to a vibrating device. (Chemapac Inc. Vibro Mixer El). Some of the ampules were vibrated, while a some of the ampules were not vibrated. Following equilibration for five, seven, nine or fifteen days, the ampules were removed and the samples were filtered through a 0.22 micrometer filter (Millipore Millex-GS (mixed esters of cellulose) into 20-mL screw top scintillation vials. The pH of the undiluted sample solutions were measured using a pH meter equipped with a combination electrode (Radiometer Model PHM85 pH Meter with High pH combination Electrode GK2402B). These samples were then diluted and assayed according to the procedure detailed below and gave the following pH-solubility profiles:

| | |
|---|---|
| pH | [M] |

5 Day Equilibrium with Vibration

| | |
|---|---|
| 1.14 | 6.18e⁻⁴ |
| 1.24 | 8.09e⁻⁴ |
| 1.46 | 1.29e⁻³ |
| 2.03 | 3.09e⁻³ |
| 2.61 | 4.48e⁻³ |
| 2.78 | 4.68e⁻³ |
| 3.29 | 4.17e⁻³ |
| 3.51 | 2.88e⁻³ |
| 3.73 | 2.01e⁻³ |
| 4.22 | 1.03e⁻³ |
| 4.54 | 6.45e⁻⁴ |
| 4.55 | 7.33e⁻⁴ |
| 5.45 | 4.92e⁻⁴ |
| 8.03 | 3.12e⁻⁴ |
| 8.03 | 3.08e⁻⁴ |
| 8.08 | 3.38e⁻⁴ |
| 8.21 | 3.09e⁻⁴ |
| 8.33 | 3.20e⁻⁴ |
| 8.42 | 3.08e⁻⁴ |
| 8.46 | 3.08e⁻⁴ |
| 9.44 | 6.01e⁻⁴ |
| 10.17 | 1.95e⁻³ |
| 10.91 | 0.02 |

5 Day Equilibrium (No Vibration)

| | |
|---|---|
| 1.24 | 7.49e⁻⁴ |
| 1.43 | 1.04e⁻³ |
| 1.86 | 2.39e⁻³ |
| 2.53 | 5.07e⁻³ |
| 3.51 | 2.12e⁻³ |
| 3.15 | 4.31e⁻³ |
| 3.99 | 9.70e⁻⁴ |
| 4.22 | 7.21e⁻⁴ |
| 4.42 | 5.92e⁻⁴ |
| 5.87 | 3.34e⁻⁴ |

7 day Equilibrium with Vibration

| | |
|---|---|
| 2.34 | 4.41e⁻³ |
| 2.27 | 3.81e⁻³ |
| 3.29 | 3.16e⁻³ |
| 3.38 | 2.77e⁻³ |
| 4.05 | 8.97e⁻⁴ |
| 6.27 | 2.98e⁻⁴ |
| 9.86 | 1.20e⁻³ |
| 10.74 | 4.92e⁻³ |
| 11.01 | 8.40e⁻³ |
| 11.10 | 0.01 |
| 11.16 | 0.01 |
| 11.17 | 0.02 |

9 Day Equilibrium with Vibration

| | |
|---|---|
| 0.73 | 5.28e⁻⁴ |
| 1.12 | 1.10e⁻³ |
| 2.22 | 4.10e⁻³ |
| 2.96 | 3.74e⁻³ |
| 3.93 | 9.28e⁻⁴ |
| 4.60 | 5.59e⁻⁴ |
| 8.04 | 2.82e⁻⁴ |
| 9.44 | 4.73e⁻⁴ |
| 9.90 | 9.58e⁻⁴ |
| 10.09 | 1.48e⁻³ |
| 10.28 | 2.04e⁻³ |
| 10.43 | 2.71e⁻³ |
| 10.53 | 3.38e⁻³ |
| 10.67 | 4.11e⁻³ |
| 10.76 | 4.63e⁻³ |
| 10.83 | 5.35e⁻³ |
| 10.90 | 5.76e⁻³ |
| 10.99 | 6.61e⁻³ |

9 Day Equilibrium (No Vibration)

| | |
|---|---|
| 1.18 | 6.24e⁻⁴ |
| 1.43 | 9.98e⁻⁴ |
| 1.85 | 2.54e⁻³ |
| 2.50 | 4.62e⁻³ |
| 3.12 | 4.53e⁻³ |
| 3.48 | 2.12e⁻³ |
| 3.97 | 9.65e⁻⁴ |
| 4.18 | 7.42e⁻⁴ |
| 4.36 | 5.74e⁻⁴ |
| 6.00 | 3.04e⁻⁴ |

15 Day Equilibrium with Vibration

| | |
|---|---|
| 1.27 | 5.98e⁻⁴ |
| 1.58 | 1.32e⁻³ |
| 2.01 | 3.26e⁻³ |
| 2.06 | 3.28e⁻³ |
| 2.69 | 4.95e⁻³ |
| 2.83 | 3.96e⁻³ |
| 3.35 | 3.34e⁻³ |
| 3.49 | 2.87e⁻³ |
| 3.65 | 2.08e⁻³ |
| 4.06 | 1.07e⁻³ |
| 4.32 | 7.88e⁻⁴ |
| 4.51 | 6.56e⁻⁴ |
| 4.84 | 5.06e⁻⁴ |
| 5.98 | 3.67e⁻⁴ |
| 7.43 | 3.02e⁻⁴ |
| 7.51 | 3.04e⁻⁴ |
| 7.83 | 3.51e⁻⁴ |

### Example 18: Preparation/Identification of Preferred solution conformation of Form

I″4-[4-[4-(hydroxydiphenylmethyl)-1-piperidinyl]-1-hydroxybutyl]-α,α-dimethylbenzeneacetic acid Into each of fourteen to twenty-three 10-mL ampules (Wheaton Flint Glass), approximately 50.0 mg of Form I anhydrous 4-[4-[4-(hydroxydiphenylmethyl)-1-piperidinyl]-1-hydroxybutyl]-α,α-dimethylbenzeneacetic acid hydrochloride salt were weighed. To these ampules various concentrations of acid (HCI) or base (NaOH) were added ranging in concentration from 0 to 0.2 M with the total volume in each ampule being 10 mL. The ampules were then heat sealed (Cozzoli Model HS-1), placed into a 25° C. constant temperature bath controlled to within 0.01° C. (Tamson TEV-45) and attached to a vibrating device. (Chemapac Inc. Vibro Mixer E1). Some of the ampules were vibrated, while a some of the ampules were not vibrated. Following equilibration for five or nine days, the ampules were removed and the samples were filtered through a 0.22 micrometer filter (Millipore Millex-GS (mixed esters of cellulose) into 20-mL screw top scintillation vials. The pH of the undiluted sample solutions were measured using a pH meter equipped with a combination electrode (Radiometer Model PHM85 pH Meter with High pH combination Electrode GK2402B). These samples were then diluted and assayed according to the procedure detailed below and gave the following pH-solubility profiles:

| | |
|---|---|
| pH | [M] |

5 Day Equilibrium with Vibration

| | |
|---|---|
| 1.09 | 4.99e⁻⁴ |
| 1.35 | 8.68e⁻⁴ |
| 2.05 | 2.88e⁻³ |
| 2.40 | 3.95e⁻³ |
| 2.60 | 4.32e⁻³ |
| 2.64 | 3.84e⁻³ |
| 2.66 | 3.98e⁻³ |
| 2.68 | 3.97e⁻³ |
| 2.68 | 3.81e⁻³ |
| 2.75 | 3.17e⁻³ |
| 2.77 | 3.12e⁻³ |
| 2.78 | 3.25e⁻³ |
| 2.79 | 3.11e⁻³ |
| 3.29 | 1.92e⁻³ |
| 4.28 | 1.04e⁻³ |
| 6.77 | 4.83e⁻⁴ |
| 9.19 | 8.66e⁻⁴ |
| 10.00 | 1.65e⁻³ |
| 11.47 | 0.02 |
| 11.47 | 0.02 |
| 12.12 | 0.02 |

5 Day Equilibrium (No Vibration)

| | |
|---|---|
| 1.08 | 4.80e⁻⁴ |
| 1.36 | 8.70e⁻⁴ |
| 2.03 | 2.79e⁻³ |
| 2.65 | 4.39e⁻³ |
| 2.68 | 3.48e⁻³ |
| 2.77 | 3.12e⁻³ |
| 3.15 | 1.58e⁻³ |
| 4.13 | 1.25e⁻³ |
| 6.73 | 6.95e⁻⁴ |
| 7.24 | 6.12e⁻⁴ |

9 Day Equilibrium with Vibration

| | |
|---|---|
| 1.07 | 4.79e⁻⁴ |
| 1.35 | 8.47e⁻⁴ |
| 2.03 | 2.91e⁻³ |
| 2.33 | 4.10e⁻³ |
| 2.54 | 4.20e⁻³ |
| 2.57 | 3.89e⁻³ |
| 2.61 | 3.72e⁻³ |
| 2.73 | 3.11e⁻³ |
| 2.84 | 2.72e⁻³ |
| 3.07 | 2.15e⁻³ |
| 3.43 | 1.59e⁻³ |
| 4.05 | 1.07e⁻³ |
| 4.30 | 7.27e⁻⁴ |
| 4.91 | 5.21e⁻⁴ |
| 5.13 | 4.33e⁻⁴ |
| 5.49 | 3.96e⁻⁴ |
| 8.50 | 4.98e⁻⁴ |
| 9.78 | 1.16e⁻³ |
| 11.93 | 8.55e⁻³ |
| 11.24 | 0.02 |
| 12.07 | 0.02 |
| 11.97 | 0.04 |

9 Day Equilibrium (No Vibration)

| | |
|---|---|
| 1.05 | 4.71e⁻⁴ |
| 1.36 | 8.68e⁻⁴ |
| 2.05 | 2.88e⁻³ |
| 2.58 | 4.30e⁻³ |
| 2.59 | 4.01e⁻³ |
| 2.74 | 3.18e⁻³ |
| 3.02 | 2.19e⁻³ |
| 3.81 | 1.31e⁻³ |
| 5.16 | 4.82e⁻⁴ |
| 5.33 | 5.02e⁻⁴ |

### Example 19: Preparation/Identification of Preferred solution conformation of Form II″4-[4-[4-(hydroxydiphenylmethyl)-1-piperidinyl]-1-hydroxybutyl]-α, α-dimethylbenzeneacetic acid

Into each of fourteen to twenty-three 10-mL ampules (Wheaton Flint Glass), approximately 50.0 mg of Form II hydrated 4-[4-[4-(hydroxydiphenylmethyl)-1-piperidinyl]-1-hydroxybutyl]-α,α-dimethylbenzeneacetic acid hydrochloride salt were weighed. To these ampules various concentrations of acid (HCI) or base (NaOH) were added ranging in concentration from 0 to 0.2 M with the total volume in each ampule being 10 mL. The ampules were then heat sealed (Cozzoli Model HS-1), placed into a 25° C. constant temperature bath controlled to within 0.01° C. (Tamson TEV-45) and attached to a vibrating device. (Chemapac Inc. Vibro Mixer E1). Following equilibration for five or fifteen days, the ampules were removed and the samples were filtered through a 0.22 micrometer filter (Millipore Millex-GS (mixed esters of cellulose) into 20-mL screw top scintillation vials. The pH of the undiluted sample solutions were measured using a pH meter equipped with a combination electrode (Radiometer Model PHM85 pH Meter with High pH combination Electrode GK2402B). These samples were then diluted and assayed according to the procedure detailed below and gave the following pH-solubility profiles:

| | |
|---|---|
| pH | [M] |

5 Day Equilibrium with Vibration

| | |
|---|---|
| 0.93 | 2.94e⁻⁴ |
| 2.51 | 3.44e⁻³ |
| 3.25 | 5.43e⁻³ |
| 3.26 | 5.46e⁻³ |
| 3.28 | 5.38e⁻³ |
| 3.28 | 5.21e⁻³ |
| 3.31 | 5.22e⁻³ |
| 3.38 | 3.40e⁻³ |
| 3.85 | 1.81e⁻³ |
| 5.10 | 1.06e⁻³ |
| 6.80 | 1.15e⁻³ |
| 9.87 | 4.49e⁻³ |
| 10.20 | 8.35e⁻³ |
| 10.24 | 1.29e⁻² |

15 Day Equilibrium with Vibration

| | |
|---|---|
| 1.09 | 5.11e⁻⁴ |
| 1.38 | 8.56e⁻⁴ |
| 2.05 | 2.89e⁻³ |
| 2.35 | 3.97e⁻³ |
| 2.68 | 4.52e⁻³ |
| 2.74 | 4.14e⁻³ |
| 2.78 | 3.84e⁻³ |
| 2.98 | 3.18e⁻³ |
| 3.08 | 2.58e⁻³ |
| 3.43 | 1.61e⁻³ |
| 3.73 | 1.32e⁻³ |
| 4.35 | 1.11e⁻³ |
| 4.69 | 8.49e⁻⁴ |
| 4.86 | 1.47e⁻² |
| 5.43 | 7.36e⁻⁴ |
| 5.93 | 7.98e⁻⁴ |

### Assay Procedure

The assay for Examples 17, 18 and 19 is as follows:.

### Special Apparatus, Regents and Solutions

Allcott Autosampler (64 position tray)

Kratos Spectroflow 757, or 783 Absorbance Detector

ABI Spectroflow 400, or Waters 6000 Solvent Delivery System

Zorbax SB Phenyl Chromatographic Column, 5-micrometer 250 cm×4.6 mm, Frit G, purchased as a packed column

Acetonitrile, HPLC grade, Baxter

Sodium phosphate, monobasic, monohydrate

Sodium perchlorate, monohydrate

GV/WP Filter Paper, Millipore

Mobile Phase: (35/65) (v/v) acetonitrile/phosphate buffer (0.048 M sodium phosphate containing 0.006 M sodium perchloriate, pH 2.0). Nominal one liter volumes of the phosphate buffer were prepared by mixing 1000 mL water with 6.64 grams of sodium phosphate, monobasic, monohydrate and 0.84 sodium perchlorate, monohydrate. The pH was adjusted to 2.0 with 85% phosphoric acid.

The final mobile phase was prepared by adding 650 mL of the above buffer solution to 350 mL of acetonitrile, the solution was mixed thoroughly, filtered and degassed under vacuum.

Date acquisition was obtained using Peak Pro supplied by Beckman Instruments.

### Chromatographic Conditons

Flow rate: 1.5 mumin

Wavelength: 220nm

Sensitivity: 0.05 AUFS

Temperature: ambient

Sample size: 20 microliters or 10 microliters

### Preparation and Response Of The Authentic Solutions

Standard Curves were generated with the starting material, using the general procedure below.

Five to nine authentic solutions of Form I′, Form I″, or Form II″ polymorphic and pseudomorphic hydrated and anhydrous 4-[4-[4-(hydroxydiphenylmethyl)-1-piperidinyl]-1-hydroxybutyl]-α,α-dimethylbenzeneacetic acid free base and hydrochloride compounds were prepared for use as standard solutions. These solutions were used to assay their respective experimental solutions. An example for the preparation of a set of standard solutions is as follows:

For the 5 day solubility of Form II study, concentrations ranging from 2.30×10−3 mg/mL to 2.88×10−1 mg/mL, were prepared: Approximately 28.80 mg of Form II hydrated 4-[4-[4-(hydroxydiphenylmethyl)-1-piperidinyl]-1-hydroxybutyl]-α,α-dimethylbenzeneacetic acid hydrochloride salt were accurately weighed on a five place analytical balance in a 100-mL volumetric flask and diluted to volume with mobile phase. Subsequent volumetric dilutions of this solutions were made to produce the rest of the standards.

### Sample Analysis

The samples were diluted using mobile phase and volumetric glassware before being assayed by HPLC. Each sample was injected three times and the median area was used to calculate the concentration of 4-[4-[4-(hydroxydiphenylmethyl)-1-piperidinyl]-1-hydroxybutyl]-α,α-dimethylbenzeneacetic acid compounds using the appropriate standard curve.

The polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) or the preferred solution conformations derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) of this invention are useful as antihistamines, antiallergy agents and bronchodilators and may be administered alone or with suitable pharmaceutical carriers, and can be in solid or liquid form such as, tablets, capsules, powders, solutions, suspensions or emulsions.

The polymorphic or pseudomorphic hydrated or anhydrous 4-[4-[4-(hydroxydiphenylmethyl)- 1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) or the preferred solution conformations derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]- α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) of this invention can be administered orally, parenterally, for example, subcutaneously, intravenously, intramuscularly, intraperitoneally, by intranasal instillation or by application to mucous membranes, such as, that of the nose, throat and bronchial tubes, for example, in an aerosol spray containing small particles of a compound of this invention in a spray or dry powder form.

The quantity of polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) or the quantity of preferred solution conformations derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) administered will vary depending on the patient and the mode of administration and can be any effective amount. The quantity of polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) or the quantity of preferred solution conformations derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) administered may vary over a wide range to provide in a unit dosage an effective amount of from about 0.01 to 20 mg/kg of body weight of the patient per day to achieve the desired effect. For example, the desired antihistamine, antiallergy and bronchodilator effects can be obtained by consumption of a unit dosage form such as a tablet containing 1 to 500 mg of a polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) of this invention taken 1 to 4 times daily. In addition, for example, the desired antihistamine, antiallergy and bronchodilator effects can be obtained by oral, parenteral, intranasal instillation or by application to mucous membranes of a liquid formulation containing a unit dosage form of a preferred solution conformation derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, VII′″, VIII′″, IX′″, I″, II″, III″ or IV″) such liquid formulation containing 1 to 500 mg of a preferred solution conformation derived from the dissolution of any of I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) of this invention taken 1 to 4 time daily.

The solid unit dosage forms can be of the conventional type. Thus, the solid form can be a capsule which can be the ordinary gelatin type containing a polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compound of this invention and a carrier, for example, lubricants and inert fillers such as lactose, sucrose or cornstarch. In another embodiment the polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compound is tableted with conventional tablet bases such as lactose, sucrose or cornstarch or gelatin, disintegrating agents such as cornstarch, potato starch or alginic acid, and a lubricant such as stearic acid or magnesium stearate.

The polymorphic or pseudomorphic hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′, II′, III′, IV′, V′, VII′, VIII′, IX′, I, II, III or IV) or the preferred solution conformations derived from the dissolution of any of Forms I′, II′, III′, IV′, V′, VII′, VIII′, IX′, I, II, III or IV hydrated or anhydrous 4-[4-[4-(hydroxydiphenylmethyl)-1-piperidinyl]-1-hydroxybutyl]-α,α-dimethylbenzeneacetic acid free base or hydrochloride salt compounds (i.e., Forms I′″, II′″, III′″, IV′″, V′″, VII′″, VIII′″, IX′″, I″, II″, III″ or IV″) of this invention may also be administered in injectable dosages by solution or suspension of the compounds in a physiologically acceptable diluent with a pharmaceutical carrier which can be a sterile liquid such as water and oils, with or without the addition of a surfactant and other pharmaceutically acceptable adjuvants. Illustrative of oils there can be mentioned those of petroleum, animal, vegatable or synthetic origin, for example, peanut oil, soybean oil or mineral oil. In general, water, saline, aqueous dextrose and related sugar solutions and glycols such as propylene glycol or polyethylene glycol are preferred liquid carriers, particularly for injectable solutions.

For use as aerosols the compounds of this invention in solution or suspension may be packaged in a pressurized aerosol container together with suitable propellants, for example, hydrocarbon propellants such as, propane, butane or isobutane with the usual adjuvants as may be administered in a non-pressurized form such as in a nebulizer or atomizer.

The term patient as used herein is taken to mean warm blooded animals, birds, mammals, for example, humans, cats, dogs, horses, sheep, bovine cows, pigs, lambs, rats, mice and guinea pigs.

What is claimed is:

1. Form I′ hydrated 4-[4-[4-(hydroxydiphenylmethyl)-1-piperidinyl]-1-hydroxybutyl]-α,α-dimethylbenzeneacetic acid.

2. Form II′ hydrated 4-[4-[4-(hydroxydiphenylmethyl)-1-piperidinyl]-1l-hydroxybutyl]-α,α-dimethylbenzeneacetic acid.

3. Form III′ hydrated 4-[4-[4-(hydroxydiphenylmethyl)-1-piperidinyl]-1-hydroxybutyl]-α,α-dimethylbenzeneacetic acid.

4. Form IV′ hydrated 4-[4-[4-(hydroxydiphenylmethyl)-1-piperidinyl]-1-hydroxybutyl]-α,α-dimethylbenzeneacetic acid.

5. Form V′ hydrated 4-[4-[4-(hydroxydiphenylmethyl)-1-piperidinyl]-1-hydroxybutyI]-α,α-dimethylbenzeneacetic acid.

6. Form VIII′ hydrated 4-[4-[4-(hydroxydiphenylmethyl)-l -piperidinyl]-1-hydroxybutyl]-α,α-dimethylbenzeneacetic acid.

7. Form IX′ hydrated 4-[4-[4-(hydroxydiphenylmethyl)-1-piperidinyl]-1-hydroxybutyl]-α,α-dimethylbenzeneacetic acid.

8. Form VII′ anhydrous 4-[4-[4-(hydroxydiphenylmethyl)-1-piperidinyl]-1-hydroxybutyl]-α,α-dimethylbenzeneacetic acid.

9. Form I an hydrous 4-[4-[4-(hydroxydiphenylmethyl)-1-piperidinyl]-1-hydroxybutyl]-α,α-dimethylbenzeneacetic acid hydrochloride salt.

10. Form II hydrated 4-[4-[4-(hydroxydiphenylmethyl)-1-piperidinyl]-1-hydroxybutyl]-α,α-dimethylbenzeneacetic acid hydrochloride salt.

11. Form III anhydrous 4-[4-[4-(hydroxydiphenylmethyl)-1-piperidinyl]-1-hydroxybutyl]-α,α-dimethylbenzeneacetic acid hydrochloride salt.

12. Form IV hydrated 4-[4-[4-(hydroxydiphenylmethyl)-1-piperidinyl-1-hydroxybutyl]-α,α-dimethylbenzeneacetic acid hydrochloride salt.

13. Form I′″ aqueous solution conformation of 4-[4-[4-(hydroxydiphenylmethyl)-1-piperidinyl]- 1-hydroxybutyl]-α,α-dimethylbenzeneacetic acid.

14. Form I′ aqueous solution conformation of 4-[4-[4-(hydroxydiphenylmethyl)-1-piperidinyl]-1-hydroxybutyl]-α,α-dimethylbenzeneacetic acid.

15. Form II′ aqueous solution conformation of 4-[4-[4-(hydroxydiphenylmethyl)-1-piperidinyl]-1-hydroxybutyl]-α,α-dimethylbenzeneacetic acid.

16. Form I′″ aqueous solution conformation of 4-[4-[4-(hydroxydiphenylmethyl)-1-piperidinyl]- 1-hydroxybutyl]-α,α-dimethylbenzeneacetic acid formed by the process of dissolving Form I′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

17. Form II′″ aqueous solution conformation of 4-[4-[4-(hydroxydiphenylmethyl)-1-piperidinyl]-1-hydroxybutyl]-α,α-dimethylbenzeneacetic acid formed by the process of dissolving Form II′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

18. Form III′″ aqueous solution conformation of 4-[4-[4-(hydroxydiphenylmethyl)-1-piperidinyl]-1-hydroxybutyl]-α,α-dimethylbenzeneacetic acid formed by the process of dissolving Form III′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-b 13.

19. Form IV′″ aqueous solution conformation of 4-[4-[4-(hydroxydiphenylmethyl)-1-piperidinyl]-1-hydroxybutyl]-α,α-dimethylbenzeneacetic acid formed by the process of dissolving Form IV″ acid in a non-buffered aqueous solution, pH of 0.5-13.

20. Form V′″ aqueous solution conformation of 4-[4-[4-(hydroxydiphenylmethyl)-1-piperidinyl]-1-hydroxybutyl]-α,α-dimethylbenzeneacetic acid formed by the process of dissolving Form V′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

21. Form VIII′″ aqueous solution conformation of 4-[4-[4-(hydroxydiphenylmethyl)-1-piperidinyl]-1-hydroxybutyl]-α,α-dimethylbenzeneacetic acid formed by the process of dissolving Form VIII′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

22. Form IX′″ solution conformation of 4-[4-[4-(hydroxydiphenylmethyl)-1-piperidinyl]-1-hydroxybutyl]-α,α-dimethylbenzeneacetic acid formed by the process of dissolving Form IX′ hydrated 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

23. Form VII′″ solution conformation of 4-[4-[4-(hydroxydiphenylmethly)-1-piperidinyl]-1-hydroxybutyl]-α,α-dimethylbenzeneacetic acid formed by the process of dissolving Form VII′ anhydrous 4-[4-[4-(hydroxydiphenylmethyl)-1-piperidinyl]-1-hydroxybutyl]-α,α-dimethylbenzeneacetic acid in a non-buffered aqueous solution, pH of 0.5-13.

24. Form I″ solution conformation of 4-[4-[4-(hydroxydiphenylmethyl)-1-piperidinyl]-1-hydroxybutyl]-α,α-dimethylbenzeneacetic acid formed by the process of dissolving Form I anhydrous 4-[4-[4-(hydroxydiphenylmethyl)-1-piperidinyl]-1-hydroxybutyl]-α,α-dimethylbenzeneacetic acid hydrochloride salt in a non-buffered aqueous solution, pH of 0.5-13.

25. Form II″ solution conformation of 4-[4-[4-(hydroxydiphenylmethyl)-1-piperidinyl]-1-hydroxybutyl]-α,α-dimethylbenzeneacetic acid formed by the process of dissolving Form II hydrated 4-[4-[4-(hydroxydiphenylmethyl)-1-piperidinyl]-1-hydroxybutyl]-α,α-dimethylbenzeneacetic acid hydrochloride salt in a non-buffered aqueous solution, pH of 0.5-13.

26. Form III″ solution conformation of 4-[4-[4-(hydroxydiphenylmethyl)-1-piperidinyl]-1-hydroxybutyl]-α,α-dimethylbenzeneacetic acid formed by the process of dissolving Form III anhydrous 4-[4-[4-(hydroxydiphenylmethyl)-1-piperidinyl]-1-hydroxybutyl]-α,α-dimethylbenzeneacetic acid hydrochloride salt in a non-buffered aqueous solution, pH of 0.5-13.

27. Form IV″ solution conformation of hydrated 4-[4-[4-(Hydroxydiphenylmethyl)-1-piperidinyl]-1-hydroxybutyl]-α,α-dimethylbenzeneacetic acid hydrochloride salt formed by the process of dissolving Form IV hydrated 4-[4-[4-(hydroxydiphenylmethyl)-1-piperidinyl]-1-hydroxybutyl]-α,α-dimethylbenzeneacetic acid hydrochloride salt in a non-buffered aqueous solution, pH of 0.5-13.

28. A pharmaceutical composition comprising an effective antiallergic amount of a compound of claim 1 in admixture or otherwise in association with an inert carrier.

29. A pharmaceutical composition comprising an effective antiallergic amount of a compound of claim 2 in admixture or otherwise in association with an inert carrier.

30. A pharmaceutical composition comprising an effective antiallergic amount of a compound of claim 3 in admixture or otherwise in association with an inert carrier.

31. A pharmaceutical composition comprising an effective antiallergic amount of a compound of claim 4 in admixture or otherwise in association with an inert carrier.

32. A pharmaceutical composition comprising an effective antiallergic amount of a compound of claim 5 in admixture or otherwise in association with an inert carrier.

33. A pharmaceutical composition comprising an effective antiallergic amount of a compound of claim 6 in admixture or otherwise in association with an inert carrier.

34. A pharmaceutical composition comprising an effective antiallergic amount of a compound of claim 7 in admixture or otherwise in association with an inert carrier.

35. A pharmaceutical composition comprising an effective antiallergic amount of a compound of claim 8 in admixture or otherwise in association with an inert carrier.

36. A pharmaceutical composition comprising an effective antiallergic amount of a compound of claim 9 in admixture or otherwise in association with an inert carrier.

37. A pharmaceutical composition comprising an effective antiallergic amount of a compound of claim 10 in admixture or otherwise in association with an inert carrier.

38. A pharmaceutical composition comprising an effective antiallergic amount of a compound of claim 11 in admixture or otherwise in association with an inert carrier.

39. A pharmaceutical composition comprising an effective antiallergic amount of a compound of claim 12 in admixture or otherwise in association with an inert carrier.

40. A method of treating allergic reactions in a patient in need thereof which comprises administering to said patient an anti-allergically effective amount of a compound of claim 1.

41. A method of treating allergic reactions in a patient in need thereof which comprises administering to said patient an anti-allergically effective amount of a compound of claim 2.

42. A method of treating allergic reactions in a patient in need thereof which comprises administering to said patient an anti-allergically effective amount of a compound of claim 3.

43. A method of treating allergic reactions in a patient in need thereof which comprises administering to said patient an anti-allergically effective amount of a compound of claim 4.

44. A method of treating allergic reactions in a patient in need thereof which comprises administering to said patient an anti-allergically effective amount of a compound of claim 5.

45. A method of treating allergic reactions in a patient in need thereof which comprises administering to said patient an anti-allergically effective amount of a compound of claim 6.

46. A method of treating allergic reactions in a patient in need thereof which comprises administering to said patient an anti-allergically effective amount of a compound of claim 7.

47. A method of treating allergic reactions in a patient in need thereof which comprises administering to said patient an anti-allergically effective amount of a compound of claim 8.

48. A method of treating allergic reactions in a patient in need thereof which comprises administering to said patient an anti-allergically effective amount of a compound of claim 9.

49. A method of treating allergic reactions in a patient in need thereof which comprises administering to said patient an anti-allergically effective amount of a compound of claim 10.

50. A method of treating allergic reactions in a patient in need thereof which comprises administering to said patient an anti-allergically effective amount of a compound of claim 11.

51. A method of treating allergic reactions in a patient in need thereof which comprises administering to said patient an anti-allergically effective amount of a compound of claim 12.

52. A process for preparing the Form I anhydrous 4-[4-[4-(Hydroxydiphenylmethyl)-1-piperidinyl]-1-hydroxybutyl]-α,α-dimethylbenzeneacetic acid hydrochloride which comprises subjecting Form III anhydrous 4-[4-[4-(Hydroxydiphenylmethyl)-1-piperidinyl]-1-hydroxybutyl]-α,α-dimethylbenzeneacetic acid hydrochloride to a crystal digestion.

53. A process for preparing the Form II hydrated 4-[4-[4-(Hydroxydiphenylmethyl)-1-piperidinyl]-1-hydroxybutyl]-α,α-dimethylbenzeneacetic acid hydrochloride which comprises:
a) reacting ethyl 4-[4-[4-(hydroxydiphenylmethyl)-1-piperidinyl]-1-oxobutyl]-α,α-dimethylbenzeneacetate hydrochloride with an appropriate reducing agent in a suitable solvent;
b) acidifying with hydrochloric acid; and
c) adding water over a period of time ranging from 1 minute to 45 minutes at a temperature range of about −20° C. to

54. A process according to claim.53 wherein the water is added over a period of time ranging from 10 minutes to 30 minutes at a temperature range of about 0° C. to 40° C.

55. A process according to claim 54 wherein the water is added over a period of time ranging from 10 minutes to 30 minutes at a temperature range of about 20° C. to 40° C.

56. A process for preparing the Form II hydrated 4-[4-[4-(Hydroxydiphenylmethyl)-1-piperidinyl]-1-hydroxybutyl]-α,α-dimethylbenzeneacetic acid hydrochloride which comprises:
a) reacting ethyl 4-[4-[4-(hydroxydiphenylmethyl)-1-piperidinyl]-1-oxobutyl]-α,α-dimethylbenzeneacetate with an appropriate reducing agent in a suitable solvent;
b) acidifying with hydrochloric acid; and
c) adding water over a period of time ranging from 1 minute to 45 minutes at a temperature range of about −20° C. to 50° C.

57. A process according to claim 56 wherein the water is added over a period of time ranging from 10 minutes to 30 minutes at a temperature range of about 0° C. to 40° C.

58. A process according to claim 57 wherein the water is added over a period of time ranging from 10 minutes to 30 minutes at a temperature range of about 20° C. to 40° C.

59. A process for preparing the Form IV hydrated 4-[4-[4-(Hydroxydiphenylmethyl)-1-piperidinyl]-1-hydroxybutyl]-α,α-dimethylbenzeneacetic acid hydrochloride which comprises:
a) reacting ethyl 4-[4-[4-(hydroxydiphenylmethyl)-1-piperidinyl]-1-oxobutyl]-α,α-dimethylbenzeneacetate hydrochloride with an appropriate reducing agent in a suitable solvent;
b) acidifying with hydrochloric acid; and
c) adding water over a period of time ranging from 30 minutes to 24 hours at a temperature range of about 0° C. to 50° C.

60. A process according to claim 59 wherein the water is added over a period of time ranging from 1 hour to 12 hours at a temperature range of about 10° C. to 40° C.

61. A process according to claim 60 wherein the water is added over a period of time ranging from 2 hours to 8 hours at a temperature range of about 20° C. to 40° C.

62. A process for preparing the Form IV hydrated 4-[4-[4-(Hydroxydiphenylmethyl)-1-piperidinyl]-1-hydroxybutyl]-α,α-dimethylbenzeneacetic acid hydrochloride which comprises:
a) reacting ethyl 4-[4-[4-(hydroxydiphenylmethyl)-1-piperidinyl]-1-oxobutyl]-α,α-dimethylbenzeneacetate with an appropriate reducing agent in a suitable solvent;
b) acidifying with hydrochloric acid; and
c) adding water over a period of time ranging from 30 minutes to 24 hours at a temperature range of about 0° C., to 50° C.

63. A process according to claim 62 wherein the water is added over a period of time ranging from 1 hour to 12 hours at a temperature range of about 10° C. to 40° C.

64. A process according to claim 63 wherein the water is added over a period of time ranging from 2 hours to 8 hours at a temperature range of about 2000 to 40° C.
